(19) United States
(12) Patent Application Publication
(10) Pub. No.: US 2026/0008998 A1
(43) Pub. Date: Jan. 8, 2026

(54) METHODS OF MAKING AND USING IMMUNE-TOLERIZED FETAL STEM CELL-DERIVED EXTRACELLULAR VESICLES FOR HAIR LOSS TREATMENT

(71) Applicant: STEMMEDICARE CO., LTD., Seoul (KR)

(72) Inventor: Jang Ho LEE, Seoul (KR)

(73) Assignee: STEMMEDICARE CO., LTD., Seoul (KR)

(21) Appl. No.: 19/010,883

(22) Filed: Jan. 6, 2025

(30) Foreign Application Priority Data
Jul. 3, 2024 (KR) 10-2024-0087573

(51) Int. Cl. C12N 5/0735; A61K 9/50; A61K 35/545; A61P 17/14; C12N 15/113

(52) U.S. Cl. CPC C12N 5/0606; A61K 9/5068; A61K 35/545; A61P 17/14; C12N 15/113; C12N 2310/141; C12N 2502/1323; C12N 2502/1382; C12N 2527/00

(57) ABSTRACT

The use of immune-tolerized fetal stem cell-derived extracellular vesicles as a pharmaceutical composition for hair loss treatment is disclosed. The pharmaceutical composition of immune-tolerized fetal stem cell-derived extracellular vesicles for hair loss treatment contains noggin, sclerostin domain-containing protein 1 (SOSTDC1), fibroblast growth factor-7 (FGF-7), and platelet-derived growth factor-alpha (PDGF-α) to promote the conversion of hair in the catagen phase into hair in the anagen phase.

### TECHNICAL FIELD

The present invention relates to the use of immune-tolerized fetal stem cell-derived extracellular vesicles as a pharmaceutical composition for hair loss treatment, a method for manufacturing immune-tolerized fetal stem cell-derived extracellular vesicles for hair loss treatment, and the use of a cosmetic composition for hair regeneration and hair loss alleviation containing immune-tolerized fetal stem cell-derived extracellular vesicles for hair loss treatment. In particular, the present invention relates to a pharmaceutical composition for hair loss treatment containing immune-tolerized fetal stem cell-derived extracellular vesicles, which have the efficacy of improving blood circulation through the promotion of angiogenesis and the efficacy of promoting the conversion of hair in the catagen and telogen phases into normal hair in the anagen phase, and prevent immune rejection due to allogeneic transplantation and a manufacturing method thereof, and a cosmetic composition for hair regeneration and hair loss alleviation containing immune-tolerized fetal stem cell-derived extracellular vesicles.

### BACKGROUND ART

The present invention was completed with the support of the Ministry of SMEs and Startups of the Republic of Korea under Research and Development Project No. RS-2022-T1022662, titled “Development of functional cosmetics to help alleviate hair loss symptoms using extracellular vesicles that have the effect of improving blood circulation and promoting hair cycle normalization”.

The information contained in this background art section has been prepared to enhance the understanding of the background of the invention and may include matters that are not prior art already known to those skilled in the art to which this technology belongs.

It is known that there are about 5 million hairs on the human body, and it is estimated that more than 100,000 of them are on the head. The hair density is approximately 1,000 to 1,100 hairs/cm2, and no new hair follicles are formed after birth, but it gradually decreases with age, and the hair density of Westerners in their 20s and 30s is known to be around 200 to 250 hairs/cm2.

Human hair continuously grows and falls out through its unique growth cycle. The growth cycle is divided into the anagen phase, the catagen phase, and the telogen phase. The anagen phase lasts from 2 to 6 years, during which hair grows and accounts for most (85% to 90%) of the scalp hair. The catagen phase lasts for 1 to 2 weeks, during which hair growth stops and the hair bulb degenerates. The telogen phase lasts for 5 to 6 weeks, during which the hair follicle becomes inactive and hair falls out. Normally, 50 to 100 hairs fall out per day, but alopecia is suspected if more than 100 hairs consistently fall out every day.

Hair loss refers to a condition in which hair is absent or insufficient in sites where hair should normally exist, and generally refers to the loss of terminal hair (coarse, dark hair) on the scalp. Hair growth may be affected by various hormones, nutrients, and medications, and male hormones, especially testosterone, are known to significantly affect.

The most common types of hair loss include female pattern hair loss, alopecia areata, telogen effluvium, and anagen effluvium in addition to androgenetic alopecia (AGA) caused by male hormones and genetic factors. Sometimes there may be hair loss associated with scalp infections, autoimmune diseases, scalp tumors and the like.

AGA, which is caused by male hormones and genetics, occurs in about 50% of both men and women, and usually begins in the 40s and 50s, but may begin shortly after puberty and persist for decades in severe cases.

The main target of AGA is known to be dihydrotestosterone (DHT), which is converted from the male hormone testosterone by 5-alpha reductase (5AR). It has been found that DHT has an affinity for androgen receptors, which is 20 to 30 times stronger than that of testosterone and adrenal androgens, and thus causes hair loss by damaging Wnt/β-catenin signaling, which is essential for hair follicle neogenesis, development, and maintenance.

A number of therapeutic agents for hair loss are being developed targeting DHT, and Finasteride, an oral therapeutic agent for AGA approved by the US FDA, and Dutasteride, the first therapeutic agent for hair loss approved in Korea, are inhibitors of 5AR that converts testosterone into DHT, but can only be used by adult males due to the side effect of causing abnormal development of the male fetus's genitals when used by pregnant or potentially pregnant women, and may also cause erectile dysfunction and decreased libido in men.

Meanwhile, Minoxidil, which was used as a therapeutic agent for high blood pressure and was approved as a therapeutic agent for hair loss in 1988, is the only topical therapeutic agent known to be effective in treating hair loss by increasing blood flow to the scalp, but causes discomfort such as itchiness, stinging, and flaking at the site of topical application. Minoxidil has recently been approved for oral use, but side effects such as systemic hirsutism, leg edema, and orthostatic hypotension have been reported.

Minoxidil should also not be used by women who may be pregnant or breastfeeding, by those under 18 years of age, or by those with inflammation or infection on the scalp. Minoxidil is not recommended for use in cases of sudden partial hair loss, such as immediately after childbirth, or hair loss unrelated to genetic causes.

In addition to approved therapeutic agents for hair loss, hair transplantation, laser surgery, and various products including oral medications, and functional cosmetics for hair loss improvement are being developed and sold. However, there is currently no product or therapeutic agent that has been scientifically proven to be effective and can be safely used by all patients with hair loss, regardless of sex or age, without side effects.

Recently, the development of therapeutic agents for hair loss using stem cells that can differentiate into various cell types has been actively pursued. Initially, autologous adipose-derived stem cell transplantation was mainly performed because of immune rejection, but because of the decrease in effect due to high cost and low engraftment rate of stem cell therapy, the development of a therapeutic agent for hair loss using a stem cell culture solution containing various growth factors secreted from the stem cell has attracted attention and four global clinical trials are in progress. Recently, therapeutic agents for hair loss using extracellular vesicles (EVs) with high purity isolated from stem cell culture solutions are being actively developed.

Extracellular vesicles, commonly known as exosomes, are nano-sized vesicles with a double lipid membrane structure that are secreted outside of living cells, contain various physiologically active substances, such as proteins, enzymes, and nucleic acids, and play a role in delivering the substances to other cells or tissues.

According to recently published study results, stem cell-derived extracellular vesicles can contribute to hair growth by promoting the proliferation and preventing the apoptosis of dermal papilla cells (DPCs) and hair follicle stem cells (HFSCs), which play an important role in hair growth and cycle regulation.

Stem cell-derived extracellular vesicles are also known to accelerate the hair cycle transition from the telogen phase to the anagen phase by activating the Wnt/β-catenin pathway. Some studies have reported the hair loss-alleviating efficacy by the use of DPC-derived extracellular vesicles or bovine colostrum-derived extracellular vesicles. However, most studies have evaluated the hair loss treating efficacy through in vitro experiments using DPC or HFSC and animal experiments using mice, making it insufficient to present an accurate hair loss treatment mechanism. In particular, as with other therapeutic agents for hair loss, there is a limitation in that immunogenicity due to extracellular vesicles that require long-term repeated use has not been considered.

In relation to the hair loss treatment mechanism, DKK1 (dickkopf 1) has recently attracted attention as an inhibitor of the canonical Wnt/β-catenin pathway, a key signaling pathway for hair growth. Specifically, DKK1 negatively affects the hair cycle and hair follicle formation by potently inhibiting the activation of the Wnt/β-catenin signaling pathway by interfering with the formation of the frizzled low-density lipoprotein receptor-related proteins 5/6 complex induced by Wnt, thereby promoting the transition of the hair cycle from the anagen phase to the catagen phase (J. Cosmet. Dermatol., 2016. 15).

DKK1 also inhibits hair growth by promoting the apoptosis of hair follicle keratinocytes, key cells involved in hair growth (J. Investig. Dermatol., 2008, 128). In particular, a study showing that immunohistochemical expression of DKK1 is significantly increased in lesional scalp biopsies from both patients with AGA and patients with alopecia areata supports the idea that DKK1, whose expression is upregulated in DPCs by DHT, is a key target that induces AGA (Am. J. Dermatopathol., 2019, 41). However, no therapeutic agent for hair loss targeting DKK1 has been developed yet, and only various miRNAs that directly target DKK1 and suppress gene expression in various tissues have been discovered.

Extracellular vesicles secreted from non-autologous cells may cause an undesirable immune response in the patient, similar to allogeneic cell therapy, due to mismatch of the major histocompatibility complex (MHC) of the originating cell present in their double lipid membrane, and a report has recently been published in Japan recommending matching human leukocyte antigens (HLA) between donors and recipients or the use of immunosuppressants in clinical applications of allogeneic extracellular vesicles (Japan Pharmaceuticals and Medical Devices Agency (PMDA), January 2023).

T. Driedonks et al. have reported that the blood half-life of administered allogeneic extracellular vesicles gradually decreases by the activation of adaptive immunity to repeatedly administered allogeneic extracellular vesicles. The blood half-life decreases by approximately 75% or more compared to that in the first administration when allogeneic extracellular vesicles are administered 4 to 5 times, and the production of immunoglobulins specific to the administered allogeneic extracellular vesicles also gradually increases (J. Extracell. Bio., 2022. 10).

### CITATION LIST

### Patent Documents

Patent Document 1: U.S. Ser. No. 11/566,220 B2
Patent Document 2: U.S. Ser. No. 11/771,720 B2
Patent Document 3: U.S. Ser. No. 12/150,962 B2

### DISCLOSURE

### Technical Problem

An object of the present invention is to provide a pharmaceutical composition for hair loss treatment containing immune-tolerized fetal stem cell-derived extracellular vesicles, which contain miRNA components that suppress the expression of the DKK1 gene, which induces hair loss, components that induce hair follicle formation and development, and transition to the anagen phase by inhibiting BMP signaling, which maintains HFSCs in a quiescent state, inhibits differentiation into hair follicle cells, and maintains quiescent hair follicles, and activating Wnt/β-catenin signaling, and components that promote the improvement of blood circulation at much higher levels compared to fetal stem cell-derived extracellular vesicles obtained under general culture conditions, and have superior efficacy in normalizing the hair cycle and alleviating hair loss; a method for manufacturing immune-tolerized fetal stem cell-derived extracellular vesicles for hair loss treatment; and a cosmetic composition for hair regeneration and hair loss alleviation containing immune-tolerized fetal stem cell-derived extracellular vesicles for hair loss treatment.

An object of the present invention is to provide a pharmaceutical composition for hair loss treatment containing immune-tolerized fetal stem cell-derived extracellular vesicles, which can deliver active ingredients completely into target cells and tissues without causing immune rejection due to antigen mismatch of allogeneic extracellular vesicles, and thus do not cause side effects when repeatedly used; a method for manufacturing immune-tolerized fetal stem cell-derived extracellular vesicles for hair loss treatment; and a cosmetic composition for hair regeneration and hair loss alleviation containing immune-tolerized fetal stem cell-derived extracellular vesicles for hair loss treatment.

### Technical Solution

A pharmaceutical composition for hair loss treatment containing immune-tolerized fetal stem cell-derived extracellular vesicles according to an embodiment of the present invention contains noggin, sclerostin domain-containing protein 1 (SOSTDC1), fibroblast growth factor-7 (FGF-7), and platelet-derived growth factor-alpha (PDGF-α) to promote the conversion of hair in the catagen phase into hair in the anagen phase.

Here, the extracellular vesicles additionally contain vascular endothelial growth factor (VEGF), fibroblast growth factor-2 (FGF-2), epidermal growth factor-like domain-containing protein 7 (EGFL7), receptor activity-modifying protein 1 (RAMP1), GATA-binding protein 2 (GATA2), chemokine (C—C motif) ligand 2 (CCL2), and RAS-interacting protein 1 (RASIP1) to promote angiogenesis.

The extracellular vesicles additionally contain miR-29, miR-203, and miR-218 to suppress DKK1 gene expression.

The extracellular vesicles are established by culturing fetal stem cells isolated from amniotic fluid collected for amniocentesis in early pregnancy in an ex vivo culture matrix that induces immune tolerance properties.

A method for manufacturing immune-tolerized fetal stem cell-derived extracellular vesicles for hair loss treatment according to an embodiment of the present invention includes: (a) obtaining extracellular vesicles containing an extracellular matrix that promotes hair follicle formation and development through the co-culture of human dermal fibroblasts and adipose-derived mesenchymal stem cells; (b) preparing an ex vivo culture matrix that induces immune tolerance properties that can continuously express and secret HLA-G protein in a target cell; and (c) inoculating fetal stem cells into the ex vivo culture matrix that induces immune tolerance properties and subculturing the cells in a serum-free medium containing the extracellular vesicles obtained through the co-culture of human dermal fibroblasts and adipose-derived mesenchymal stem cells in step (a) under temperature change and vibration culture conditions similar to those in the body during pregnancy, in which the temperature change and vibration culture conditions similar to those in the body during pregnancy in step (c) are a temperature change condition having a 5-day cycle in which the temperature ranges from 36.0° C. to 37.0° C. and a vibration culture condition having a 24-hour cycle in which the vibration ranges from 0 RPM to 60 RPM (excluding 0 RPM).

Here, the method further includes (d) inoculating the subcultured immune-tolerized fetal stem cells into a culture plate, culturing the cells in a serum-free medium, and obtaining a culture supernatant; and (e) performing multi-stage membrane filtration and tangential flow filtration on the culture supernatant to separate extracellular vesicles at a high concentration.

The extracellular vesicles obtained through the co-culture of human dermal fibroblasts and adipose-derived mesenchymal stem cells in step (a) are manufactured through (a-1) inoculating human dermal fibroblasts and adipose-derived mesenchymal stem cells onto the upper and lower parts of a co-culture plate, respectively; (a-2) performing the co-culture in a serum-free medium and obtaining a culture supernatant; and (a-3) performing multi-stage filtration and separation on the culture supernatant.

The extracellular vesicles obtained through the co-culture of human dermal fibroblasts and adipose-derived mesenchymal stem cells in step (a) contain collagen types IV, VI and VII, laminins and integrins, which are extracellular matrix components that induce secretion of factors necessary for hair follicle formation and development, hair follicle stem cell activation, and hair cycle transition from the catagen phase to the anagen phase, in fetal stem cells.

The ex vivo culture matrix inducing immune tolerance properties that can continuously express and secret HLA-G protein in a target cell in step (b) is manufactured through (b-1) obtaining extracellular vesicles through the co-culture of human amniotic membrane-derived mesenchymal stem cells and amniotic fluid-derived mesenchymal stem cells; (b-2) inoculating human trophoblasts into an ex vivo culture gel containing hyaluronic acid and the extracellular vesicles obtained in stem (b-1); (b-3) culturing the trophoblasts under temperature change and vibration culture conditions similar to those in the body during pregnancy; (b-4) obtaining immune-tolerized trophoblast-derived extracellular vesicles through serum-free culture of the trophoblasts with induced immune tolerance properties; and (b-5) adding hyaluronic acid to each of the extracellular vesicles obtained in step (b-1) and the extracellular vesicles obtained in step (b-4) and mixing.

The ex vivo culture gel in step (b-1) and the ex vivo culture matrix in step (c) are maintained in an acidic condition of pH 6 to 7.

The subcultured fetal stem cells of step (d) are fetal stem cells with induced immune tolerance properties that can express HLA-G protein on the cell surface, secrete HLA-G protein outside the cell, and HLA-G protein is present in the culture supernatant.

The obtained immune-tolerized fetal stem cell-derived extracellular vesicles reduce gene expression of BMP2, BMP4, BMP6, DKK1, DKK2, and DKK3, which maintain the hair catagen phase in human hair follicle dermal papilla cells and induce hair loss, thereby alleviating hair loss symptoms.

The obtained immune-tolerized fetal stem cell-derived extracellular vesicles increase gene expression of WNT10B, WNT16, WNT5A, WNT5B, and β-catenin, which induce the anagen phase in human hair follicle dermal papilla cells and promote hair follicle growth and differentiation, thereby alleviating hair loss symptoms.

Immune-tolerized fetal stem cell-derived extracellular vesicles for hair loss treatment that are manufactured by the method are used in a cosmetic composition for hair regeneration and hair loss alleviation containing immune-tolerized fetal stem cell-derived extracellular vesicles for hair loss treatment according to an embodiment of the present invention.

### Advantageous Effects

According to the present invention, the immune-tolerized fetal stem cell-derived extracellular vesicles for hair loss treatment contain miRNA components that suppress the expression of the DKK1 gene, which induces hair loss, components that induce hair follicle formation and development, and transition to the anagen phase by inhibiting BMP signaling, which maintains HFSCs in a quiescent state, inhibits differentiation into hair follicle cells, and maintains quiescent hair follicles, and activating Wnt/β-catenin signaling, and components that promote improvement of blood circulation at much higher levels compared to fetal stem cell-derived extracellular vesicles obtained under general culture conditions, and have superior efficacy in normalizing the hair cycle and alleviating hair loss.

According to the present invention, the immune-tolerized fetal stem cell-derived extracellular vesicles for hair loss treatment can be used as a composition for hair loss treatment, which can deliver active ingredients completely into target cells and tissues without causing immune rejection due to antigen mismatch of allogeneic extracellular vesicles and does not cause side effects when repeatedly used.

### BRIEF DESCRIPTION OF DRAWINGS

FIG. 1 is a schematic diagram illustrating a manufacturing process of immune-tolerized fetal stem cell-derived extracellular vesicles (itHT-FSC-EVs) according to an embodiment of the present invention;

FIG. 2 illustrates the results of diluting immune-tolerized fetal stem cell-derived extracellular vesicles for hair loss treatment (itHT-FSC-EVs) before a tangential flow filtration process according to an embodiment of the present invention 10-fold and analyzing the particle number concentration and size distribution of the extracellular vesicles by nanoparticle tracking analysis (NTA);

FIG. 3 illustrates the results of diluting immune-tolerized fetal stem cell-derived extracellular vesicles for hair loss treatment (itHT-FSC-EVs) before a tangential flow filtration process according to an embodiment of the present invention 100-fold and analyzing the particle number concentration and size distribution of the extracellular vesicles by nanoparticle tracking analysis (NTA);

FIG. 4 illustrates the results of diluting one lot of immune-tolerized fetal stem cell-derived extracellular vesicles for hair loss treatment (itHT-FSC-EVs) after a tangential flow filtration process according to an embodiment of the present invention 100-fold and analyzing the particle number concentration and size distribution of the extracellular vesicles by nanoparticle tracking analysis (NTA);

FIG. 5 illustrates the results of diluting another lot of immune-tolerized fetal stem cell-derived extracellular vesicles for hair loss treatment (itHT-FSC-EVs) after a tangential flow filtration process according to an embodiment of the present invention 100-fold and analyzing the particle number concentration and size distribution of the extracellular vesicles by nanoparticle tracking analysis (NTA);

FIG. 6 is a graph illustrating the results of analyzing expression levels of genes, which promote angiogenesis, contained in immune-tolerized fetal stem cell-derived extracellular vesicles for hair loss treatment (itHT-FSC-EVs) according to an embodiment of the present invention, immune-tolerized fetal stem cell-derived extracellular vesicles (itFSC-EVs), and human adipose mesenchymal stem cell-derived extracellular vesicles (AD-MSC-EVs) by next generation sequencing (NGS);

FIG. 7 is a graph illustrating the results of analyzing expression levels of genes, which are related to hair cycle normalization, contained in immune-tolerized fetal stem cell-derived extracellular vesicles for hair loss treatment (itHT-FSC-EVs) according to an embodiment of the present invention, immune-tolerized fetal stem cell-derived extracellular vesicles (itFSC-EVs), and human adipose mesenchymal stem cell-derived extracellular vesicles (AD-MSC-EVs) by next generation sequencing (NGS);

FIG. 8 is a graph illustrating the results of analyzing expression levels of microRNAs (miRNAs), which suppress the DKK1 gene expression, contained in immune-tolerized fetal stem cell-derived extracellular vesicles for hair loss treatment (itHT-FSC-EVs) according to an embodiment of the present invention, immune-tolerized fetal stem cell-derived extracellular vesicles (itFSC-EVs), and human adipose mesenchymal stem cell-derived extracellular vesicles (AD-MSC-EVs) by next generation sequencing (NGS);

FIG. 9 is microscopic images of human umbilical vein endothelial cells (HUVECs) taken 2 hours and 6 hours after treating with minoxidil and itHT-FSC-EVs at different concentrations to evaluate the angiogenesis promoting efficacy of immune-tolerized fetal stem cell-derived extracellular vesicles for hair loss treatment (itHT-FSC-EVs) according to an embodiment of the present invention;

FIG. 10 is a graph illustrating the results of analyzing the microscopic images in FIG. 9 using ImageJ software to quantify the angiogenesis promoting efficacy of immune-tolerized fetal stem cell-derived extracellular vesicles for hair loss treatment (itHT-FSC-EVs) according to an embodiment of the present invention;

FIG. 11 is a graph illustrating the results of analyzing the contents of genes that maintain the hair catagen phase and prolong hair loss in HFDPCs in the catagen phase induced by DKK1 two days after treating with minoxidil, FGF-12, and itHT-FSC-EVs at different concentrations by next generation sequencing (NGS) to evaluate the hair cycle normalizing efficacy of immune-tolerized fetal stem cell-derived extracellular vesicles for hair loss treatment (itHT-FSC-EVs) according to an embodiment of the present invention on human follicle dermal papilla cells;

FIG. 12 is a graph illustrating the results of analyzing the contents of genes that promote the transition to the anagen phase and induce hair growth and differentiation to alleviate hair loss in HFDPCs in the catagen phase induced by DKK1 two days after treating with minoxidil, FGF-12, and itHT-FSC-EVs at different concentrations by next generation sequencing (NGS) to evaluate the hair cycle normalizing efficacy of immune-tolerized fetal stem cell-derived extracellular vesicles for hair loss treatment (itHT-FSC-EVs) according to an embodiment of the present invention on human follicle dermal papilla cells;

FIG. 13 is a graph illustrating the results of evaluating the efficacy of alleviating hair loss symptoms through the measurement of the number of hairs at 8, 16, and 24 weeks after daily application of a topical agent that contains itHT-FSC-EVs and the same topical agent that does not contain itHT-FSC-EVs for 24 weeks respectively on research subjects diagnosed with alopecia to evaluate the efficacy of alleviating hair loss symptoms of immune-tolerized fetal stem cell-derived extracellular vesicles for hair loss treatment (itHT-FSC-EVs) according to an embodiment of the present invention;

FIG. 14 is a graph illustrating the results of evaluating the efficacy of alleviating hair loss symptoms through the analysis of the improvement rate of the number of hairs at 8, 16, and 24 weeks after daily application of a topical agent that contains itHT-FSC-EVs and the same topical agent that does not contain itHT-FSC-EVs for 24 weeks respectively on research subjects diagnosed with alopecia to evaluate the efficacy of alleviating hair loss symptoms of immune-tolerized fetal stem cell-derived extracellular vesicles for hair loss treatment (itHT-FSC-EVs) according to an embodiment of the present invention;

FIG. 15 is a graph illustrating the results of evaluating the efficacy of improving the number of lost hairs through the measurement of the number of lost hairs at 8, 16, and 24 weeks after daily application of a topical agent that contains itHT-FSC-EVs and the same topical agent that does not contain itHT-FSC-EVs for 24 weeks respectively on research subjects diagnosed with alopecia to evaluate the efficacy of improving the number of lost hairs of immune-tolerized fetal stem cell-derived extracellular vesicles for hair loss treatment (itHT-FSC-EVs) according to an embodiment of the present invention;

FIG. 16 is a graph illustrating the results of evaluating the efficacy of improving the number of lost hairs through the analysis of the improvement rate of the number of lost hairs at 8, 16, and 24 weeks after daily application of a topical agent that contains itHT-FSC-EVs and the same topical agent that does not contain itHT-FSC-EVs for 24 weeks respectively on research subjects diagnosed with alopecia to evaluate the efficacy of improving the number of lost hairs of immune-tolerized fetal stem cell-derived extracellular vesicles for hair loss treatment (itHT-FSC-EVs) according to an embodiment of the present invention;

FIG. 17 is a graph illustrating the results of evaluating the hair cycle normalizing efficacy through the measurement of the number of hairs in the growth phase (anagen) at 8, 16, and 24 weeks after daily application of a topical agent that contains itHT-FSC-EVs and the same topical agent that does not contain itHT-FSC-EVs for 24 weeks respectively on research subjects diagnosed with alopecia to evaluate the hair cycle normalizing efficacy of immune-tolerized fetal stem cell-derived extracellular vesicles for hair loss treatment (itHT-FSC-EVs) according to an embodiment of the present invention;

FIG. 18 is a graph illustrating the results of evaluating the hair cycle normalizing efficacy through the analysis of the improvement rate of the number of hairs in the growth phase (anagen) at 8, 16, and 24 weeks after daily application of a topical agent that contains itHT-FSC-EVs and the same topical agent that does not contain itHT-FSC-EVs for 24 weeks respectively on research subjects diagnosed with alopecia to evaluate the hair cycle normalizing efficacy of immune-tolerized fetal stem cell-derived extracellular vesicles for hair loss treatment (itHT-FSC-EVs) according to an embodiment of the present invention;

FIG. 19 is a graph illustrating the results of evaluating the hair cycle normalizing efficacy through the measurement of the number of hairs in the death phase (telogen and catagen) at 8, 16, and 24 weeks after daily application of a topical agent that contains itHT-FSC-EVs and the same topical agent that does not contain itHT-FSC-EVs for 24 weeks respectively on research subjects diagnosed with alopecia to evaluate the hair cycle normalizing efficacy of immune-tolerized fetal stem cell-derived extracellular vesicles for hair loss treatment (itHT-FSC-EVs) according to an embodiment of the present invention; and

FIG. 20 is a graph illustrating the results of evaluating the hair cycle normalizing efficacy through the analysis of the improvement rate of the number of hairs in the death phase (telogen and catagen) at 8, 16, and 24 weeks after daily application of a topical agent that contains itHT-FSC-EVs and the same topical agent that does not contain itHT-FSC-EVs for 24 weeks respectively on research subjects diagnosed with alopecia to evaluate the hair cycle normalizing efficacy of immune-tolerized fetal stem cell-derived extracellular vesicles for hair loss treatment (itHT-FSC-EVs) according to an embodiment of the present invention.

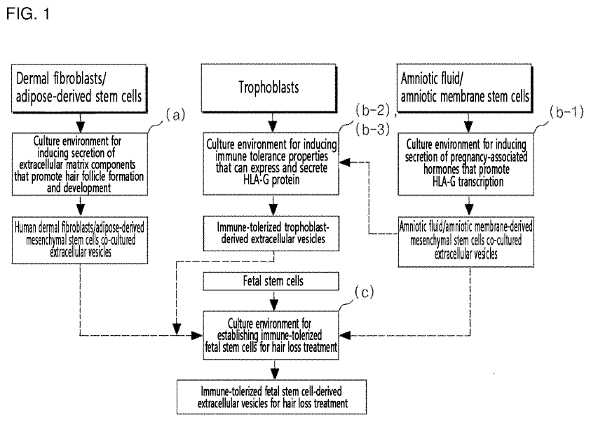

### DETAILED DESCRIPTION OF THE INVENTION

The advantages and features of the present invention and the methods for achieving them will become apparent with reference to the embodiments described below together with the accompanying drawings. However, the present invention is not limited to the embodiments disclosed below and may be implemented in various different forms. These embodiments are provided solely to ensure that the disclosure of the present invention is complete and to fully inform those skilled in the art of the scope of the invention, and the present invention is defined solely by the scope of the claims.

An embodiment of the present invention provides immune-tolerized fetal stem cell-derived extracellular vesicles for hair loss treatment, which have the efficacy of promoting angiogenesis and normalizing the hair cycle. The fetal stem cell-derived extracellular vesicles of the present invention are cultured by applying ex vivo culture conditions that simulate the in vivo environment during pregnancy, thereby containing HLA-G protein and exhibiting immune tolerance properties.

The term “extracellular vesicle” of the present invention refers to a vesicle that is produced in a cell and secreted outside the cell, and includes exosomes, microvesicles, microparticles and the like, but is not limited thereto.

In the present invention, the immune-tolerized fetal stem cell-derived extracellular vesicles for hair loss treatment may contain various components having the efficacy of promoting angiogenesis and normalizing the hair cycle.

Specifically, the immune-tolerized fetal stem cell-derived extracellular vesicles for hair loss treatment, which have angiogenesis-promoting efficacy, may contain VEGF, FGF-2, EGFL7, RAMP1, GATA2, CCL2, and RASIP1, which promote the improvement of blood circulation through angiogenesis.

The term “vascular endothelial growth factor (VEGF)” of the present invention can promote the proliferation of vascular endothelial cells and plays an important role in the regeneration of damaged blood vessels and angiogenesis.

The term “fibroblast growth factor-2 (FGF-2)” of the present invention is also called basic fibroblast growth factor (bFGF), and can contribute to angiogenesis by inhibiting the apoptosis of endothelial cells and promoting the expression of VEGF-receptor 1 (VEGFR1).

The term “epidermal growth factor-like domain-containing protein 7 (EGFL7)” of the present invention refers to an angiogenic factor mainly expressed in endothelial cells, and can contribute to angiogenesis by binding to components of the extracellular matrix.

The term “receptor activity-modifying protein 1 (RAMP1)” of the present invention can contribute to angiogenesis by promoting VEGF and VEGFR expression.

The term “GATA-binding protein 2 (GATA2)” of the present invention can play a role in promoting angiogenesis by regulating the expression of various miRNAs, including miR-126 and miR-221, which target genes directly involved in angiogenesis.

The term “chemokine (C—C motif) ligand 2 (CCL2)” of the present invention can promote the expression of proteins related to the PI3K/AKT and Wnt/β-catenin signaling pathways and play a role in promoting the proliferation, migration, and angiogenesis of human umbilical vein endothelial cells (HUVECs).

The term “RAS-interacting protein 1 (RASIP1)” of the present invention refers to a protein essential for embryonic angiogenesis, expansion, and remodeling in the second trimester of pregnancy, and can play an essential role in the angiogenesis of human endothelial cells and subcutaneous vasculature, since RASIP1 deficiency causes vascular instability and angiogenic defects.

The immune-tolerized fetal stem cell-derived extracellular vesicles for hair loss treatment, which have hair-cycle-normalizing efficacy, of the present invention may additionally contain Noggin, SOSTDC1, FGF-7, and PDGF-α that induce hair follicle formation and development and transition to the anagen phase by inhibiting BMP signaling that maintains HFSCs in a quiescent state, inhibits differentiation into hair follicle cells, and maintains quiescent hair follicles, and activating Wnt/β-catenin signaling.

The term “noggin” of the present invention refers to a protein involved in the development of a number of body tissues, including nerve tissue, muscle, and bone, and is known as an inhibitor of several bone morphogenetic proteins (BMPs), and in particular, it can contribute to hair follicle formation and activation of the postnatal hair growth stage by inhibiting BMP4.

The term “sclerostin domain-containing protein 1 (SOSTDC1)” of the present invention can contribute to promoting the conversion of hair to the anagen phase by directly binding to BMPs and inhibiting the binding of BMPs to their receptor.

The term “fibroblast growth factor-7 (FGF-7)” of the present invention is also called keratinocyte growth factor (KGF), and can contribute to promoting the proliferation of DPCs and HFSCs through the Wnt signaling pathway and inducing the differentiation of HFSCs, thereby promoting the conversion of hair to the anagen phase.

The term “platelet-derived growth factor-alpha (PDGF-α)” of the present invention is a growth factor secreted from adipocyte precursor cells, and can contribute to promoting the conversion of hair to the anagen phase by stimulating DPCs.

The immune-tolerized fetal stem cell-derived extracellular vesicles for hair loss treatment may additionally contain various microRNAs (miRs), miR-29, miR-203, and miR-218, which inhibit DKK-1 protein translation.

The term “microRNA (miR)” of the present invention refers to a short non-coding RNA molecule composed of about 22 nucleotides found in plants, animals, and microorganisms, and can play a role in regulating gene expression by binding to complementary base pairs in an mRNA molecule.

The term “miR-29” of the present invention is a microRNA that regulates the expression of DKK1, a major antagonist of Wnt signaling, kringle containing transmembrane protein 2 (KRM2), and secreted frizzled related protein 2 (SFRP2) genes. KRM2 is a transmembrane receptor with high affinity for DKK1 protein, and SFRP2 encodes a member of the SFRP family containing a domain similar to the Wnt binding site, thereby playing a role in regulating Wnt signaling. Therefore, upregulation of miR-29 can contribute to hair follicle cell regeneration and hair cycle transition by enhancing Wnt/β-catenin signaling.

The term “miR-203” of the present invention is one of the most abundantly expressed microRNAs in the epidermis and is a microRNA that binds to DKK1 mRNA and directly regulates the expression of the DKK1 gene, and upregulation of miR-203 can promote hair growth by activating Wnt/β-catenin signaling and inducing the anagen phase.

The term “miR-218” of the present invention is a microRNA that regulates the expression of the SFRP2 gene, and upregulation of miR-218 can contribute to the regeneration of hair follicle cells and the transition to the normal hair cycle by enhancing Wnt/β-catenin signaling.

The immune-tolerized fetal stem cell-derived extracellular vesicles for hair loss treatment of the present invention may additionally contain progesterone and HLA-G protein.

The term “HLA-G” of the present invention refers to a protein called human leukocyte antigen G or HLA-G histocompatibility antigen class, G, and the like. HLA-G was first discovered in extravillous trophoblasts (EVT) present at the maternal-fetal interface during pregnancy, and exists as a heterologous form that is expressed only on the cell membrane (membrane-bound HLA-G, including HLA-G1, G2, G3, and G4) or a soluble form that can be secreted outside the cell in the form of a single molecule (soluble HLA-G, including HLA-G5, G6, and G7) by alternative splicing of HLA-G mRNA.

In the present invention, HLA-G protein reduces the cytotoxicity of immune cells and promotes the differentiation into regulatory T cells due to its low polymorphism and specificity for acting on immune cells, and thus plays an essential role in establishing an immune tolerance environment that protects the fetus from the maternal immune system, especially during pregnancy.

Another embodiment of the present invention provides a method for manufacturing immune-tolerized fetal stem cell-derived extracellular vesicles for hair loss treatment, which includes (a) obtaining extracellular vesicles containing components essential for hair follicle formation and development through the co-culture of human dermal fibroblasts and adipose-derived mesenchymal stem cells; (b) preparing an ex vivo culture matrix that induces immune tolerance properties that can continuously express and secrete HLA-G protein in a target cell; and (c) inoculating fetal stem cells into the ex vivo culture matrix that induces immune tolerance properties and subculturing the cells in a serum-free medium containing the extracellular vesicles obtained through the co-culture of human dermal fibroblasts and adipose-derived mesenchymal stem cells in step (a) under temperature change and vibration culture conditions similar to those in the body during pregnancy.

The term “trophoblast” of the present invention refers to a type of cell that forms the placenta, and may refer to a cell that provides signal transmission and nutrients related to embryonic development to the inner cell mass in the early stage of development, induces successful implantation by creating immune tolerance environment that protects the fertilized embryo from the mother's immune system during the early stage of implantation, and thereafter plays an important role in the maintenance of pregnancy and development of the fetus by forming the placenta and continuously expressing and secreting the HLA-G protein.

During pregnancy, the fetus exists in amniotic fluid surrounded by the amniotic membrane, and the fetal membrane is composed of the amnion and the chorion containing trophoblasts, and the placenta is composed of the fetal chorion and the maternal decidual.

The present inventor has developed an ex vivo culture matrix (MBTC-Matrix®) that induces immune tolerance properties that can continuously express and secrete HLA-G protein in target cells by culturing human trophoblasts in an ex vivo culture gel containing extracellular vesicles obtained through the co-culture of human amniotic fluid and amniotic membrane derived stem cells and hyaluronic acid to simulate the fetal membrane structure during pregnancy in vitro and thus containing the immune-tolerized trophoblast-derived extracellular vesicles with induced immune tolerance properties in the ex vivo culture gel through prior patents, and have applied the ex vivo culture matrix to the present patent application as well (U.S. Ser. Nos. 11/566,220 B2, 11/771,720 B2, and 12/150,962 B2).

Fetal stem cells have been inoculated into the ex vivo culture matrix (MBTC-Matrix®), which induces the immune tolerance properties, thus cultured in a serum-free medium containing the extracellular vesicles obtained through the co-culture of human dermal fibroblasts and adipose-derived mesenchymal stem cells under temperature change and vibration culture conditions similar to those in the body during pregnancy to manufacture immune-tolerized fetal stem cell-derived extracellular vesicles with hair loss treating efficacy.

The method for manufacturing immune-tolerized fetal stem cell-derived extracellular vesicles for hair loss treatment having the efficacy of promoting angiogenesis and normalizing the hair cycle may further include (d) inoculating the subcultured immune-tolerized fetal stem cells into a culture plate, culturing the cells in a serum-free medium, and obtaining a culture supernatant; and (e) performing multi-stage membrane filtration and tangential flow filtration on the culture supernatant to separate extracellular vesicles at a high concentration.

The extracellular vesicles obtained through the co-culture of human dermal fibroblasts and adipose-derived mesenchymal stem cells in step (a) of the manufacturing method may be manufactured through (a-1) inoculating human dermal fibroblasts and adipose-derived mesenchymal stem cells onto the upper and lower parts of a co-culture plate, respectively; (a-2) performing the co-culture in a serum-free medium and obtaining a culture supernatant; and (a-3) performing multi-stage filtration and separation on the culture supernatant.

Specifically, step (a-1) may include (a-1-1) cryopreserving human dermal fibroblasts and adipose-derived mesenchymal stem cells in a cryopreservation composition (MBTC-CRYOGUARD®) containing immune-tolerized human mesenchymal stem cell-derived extracellular vesicles at 5% to 50% (v/v); and (a-1-2) thawing the frozen human dermal fibroblasts and adipose-derived mesenchymal stem cells and inoculating the cells onto the upper and lower parts of the co-culture plate, respectively, but is not limited thereto.

Specifically, in step (a-1-2), human dermal fibroblasts and adipose-derived mesenchymal stem cells may be inoculated at a density of 5,000 to 20,000 cells/cm2, respectively, but the inoculation density is not limited thereto.

The co-culture in step (a-1) may be performed for 100 to 140 hours, specifically 114 to 126 hours, but is not limited thereto.

In step (a-3), the culture supernatant may be separated 1 to 10 times, preferably 4 times using a filter of 0.3 μm to 1 μm, specifically 0.45 μm to 0.8 μm. In a specific example, the culture supernatant may be separated two times using a 0.8 μm filter and a 0.45 μm filter, but is not limited thereto.

The extracellular vesicles obtained through the co-culture of human dermal fibroblasts and adipose-derived mesenchymal stem cells in step (a) may contain various components that induce secretion of factors necessary for hair follicle formation and development, activation of hair follicle stem cells, and the transition of the hair cycle from the catagen phase to the anagen phase, from fetal stem cells.

The ex vivo culture matrix (MBTC-Matrix®) inducing immune tolerance properties that can continuously express and secrete HLA-G protein in a target cell of step (b) may be manufactured through (b-1) obtaining extracellular vesicles through the co-culture of human amniotic membrane-derived mesenchymal stem cells and amniotic fluid-derived mesenchymal stem cells; (b-2) inoculating human trophoblasts into an ex vivo culture gel containing hyaluronic acid and the extracellular vesicles obtained in step (b-1); (b-3) culturing the trophoblasts under temperature change and vibration culture conditions similar to those in the body during pregnancy; (b-4) obtaining immune-tolerized trophoblast-derived extracellular vesicles through serum-free culture of trophoblasts with induced immune tolerance properties; and (b-5) adding hyaluronic acid to each of the extracellular vesicles obtained in step (b-1) and the extracellular vesicles obtained in step (b-4) and performing mixing.

Specifically, step (b-1) may include (b-1-1) cryopreserving human amniotic membrane and amniotic fluid-derived mesenchymal stem cells in a cryopreservation composition (MBTC-CRYOGUARD®) containing immune-tolerized human mesenchymal stem cell-derived extracellular vesicles at 5% to 50% (v/v); (b-1-2) thawing the frozen human amniotic membrane and amniotic fluid-derived mesenchymal stem cells, inoculating the cells onto the lower and upper parts of a co-culture plate, respectively, co-culturing the cells in a serum-free medium, and obtaining a culture supernatant, but this is not limited thereto.

In step (b-2), extracellular vesicles and hyaluronic acid may be mixed at a weight ratio of 1:1 to 1:20 to manufacture the ex vivo culture gel.

In step (b-2), trophoblasts may be inoculated at a density of 5,000 to 15,000 cells/cm2.

Specifically, steps (b-1) to (b-3) may be steps of inoculating human trophoblasts into an ex vivo culture gel manufactured by mixing the extracellular vesicles obtained by multi-stage filtration and separation of the culture supernatant and hyaluronic acid, and subculturing the cells in a serum-free medium, but are not limited thereto.

(b-4) may be a step of washing the subcultured trophoblasts, inoculating the cells onto a general culture plate at a density of 10,000 to 30,000 cells/cm2, performing serum-free culture for 72 to 120 hours, and performing multi-stage filtration and separation on the obtained culture supernatant, but is not limited thereto.

At this time, the culture supernatant may be separated 1 to 10 times, preferably 4 times using a filter of 0.3 μm to 1 μm, specifically 0.45 μm to 0.8 μm. In a specific example, the culture supernatant may be separated two times using a 0.8 μm filter and a 0.45 μm filter, but is not limited thereto.

The ex vivo culture matrix (MBTC-Matrix®) that induces immune tolerance properties in target cells in (b-5) may be manufactured by mixing the extracellular vesicles obtained through the co-culture of human amniotic membrane and amniotic fluid-derived mesenchymal stem cells obtained in step (b-1), the immune-tolerized trophoblast-derived extracellular vesicles obtained in step (b-4), and hyaluronic acid at a weight ratio of 1:1:1 to 1:1:10, but is not limited thereto.

The ex vivo culture matrix (MBTC-Matrix®), which is manufactured by the method of step (b) and induces immune tolerance properties that can continuously express and secrete HLA-G protein in a target cell, may contain progesterone that promotes the gene expression of HLA-G protein that induces immune tolerance.

The ex vivo culture matrix (MBTC-Matrix®) in step (c) may maintain an acidic condition of pH 6 to 7.

The temperature change and vibration culture conditions similar to those in the body during pregnancy in step (c) may be a temperature change condition based on the basal body temperature method having a 5-day cycle in which the temperature changes in a range of 36.0° C. to 37.0° C. and a vibration culture condition having a 24-hour cycle in which the vibration changes in a range of 0 RPM to 60 RPM (excluding 0 RPM).

Specifically, the temperature change condition may be, but is not limited to, a temperature change condition in which the temperature changes to 36.5° C. at the 0 to 12th hour, 36.4° C. at the 12th to 36th hour, 36.3° C. at the 36th to 48th hour, 36.2° C. at the 48th to 60th hour, 36.0° C. at the 60th to 72nd hour, and to 37.0° C. at the 72nd to 120th hour.

Specifically, the vibration culture condition may be, but is not limited to, a vibration culture condition in which the vibration changes to 0 RPM at the 0 to 7th hour, 30 RPM at the 8th hour, 60 RPM at the 9th to 18th hour, 20 RPM at the 19th hour, and 0 RPM at the 20th to 24th hour.

The immune-tolerized fetal stem cell-derived extracellular vesicles for hair loss treatment, which are obtained through the manufacturing method, may contain various components having the efficacy of improving blood circulation through the promotion of angiogenesis and the efficacy of promoting the conversion of hair in the telogen and catagen phases into normal hair in the anagen phase.

Another embodiment of the present invention provides a pharmaceutical composition for hair loss treatment including immune-tolerized fetal stem cell-derived extracellular vesicles containing various components that promote angiogenesis and the conversion of hair to the anagen phase.

In the Examples of the present invention, when human dermal papilla cells (hDPCs) were treated with the immune-tolerized fetal stem cell-derived extracellular vesicles for hair loss treatment, the expression of DKK1, which inhibits hair growth and induces hair in the catagen phase, was suppressed, and the expression of various components that induce hair in the anagen phase was significantly increased compared to the minoxidil treatment group.

When human umbilical vein endothelial cells (HUVEC) were treated with the immune-tolerized fetal stem cell-derived extracellular vesicles for hair loss treatment, angiogenesis was significantly promoted compared to the minoxidil treatment group.

In the Examples of the present invention, when an topical agent containing the immune-tolerized fetal stem cell-derived extracellular vesicles for hair loss treatment was applied to patients with hair loss, the extracellular vesicles were found to have superior efficacy in normalizing the hair cycle and alleviating hair loss compared to the control group applying the same topical agent that did not contain the extracellular vesicles, and it has been found that the extracellular vesicles can be used for hair loss treatment.

In the present invention, the term “hair loss” or “alopecia” refers to a condition in which there is no hair in a site where hair should normally exist, and generally refers to a disease in which the terminal hair (coarse, dark hair) on the scalp falls out. Hair loss may be clinically divided into two types: scarring hair loss and non-scarring hair loss, in which scarring hair loss is hair loss where the hair follicles are destroyed and hair does not grow back and non-scarring hair loss is hair loss where the hair follicles are maintained and hair grows back after the affected site disappears. Non-scarring hair loss, which does not form scars, includes hereditary androgenetic alopecia (baldness), alopecia areata, telogen effluvium, and hair growth disorders, and scarring hair loss, which forms a scar, includes alopecia due to lupus, burns, and trauma.

Specifically, alopecia includes, but is not limited to, alopecia areata, baldness (male pattern baldness), female pattern baldness, and telogen effluvium.

The term “treatment” of the present invention means any action by which the symptoms of hair loss are improved or beneficially changed by the composition.

In the present invention, the composition is preferably used for humans, but may also be used for livestock such as cows, horses, sheep, pigs, goats, camels, antelopes, dogs or cats that may develop alopecia.

In the pharmaceutical composition for hair loss treatment of the present invention, the route and method of administration for administering the composition are not particularly limited, as any route and method of administration may be adopted as long as the composition can reach the intended site. Specifically, the composition may be administered through various routes, either orally or parenterally. Non-limiting examples of the route of administration include ocular, oral, rectal, topical, intravenous, intraperitoneal, intramuscular, intraarterial, transdermal, or intranasal administration or administration through inhalation. The composition may be administered by any device capable of transporting the active substance to the target cell.

In the present invention, the pharmaceutical composition for hair loss treatment may additionally contain a pharmaceutically acceptable carrier, excipient or diluent commonly used in the manufacture of pharmaceutical compositions, and the carrier may include a non-naturally occurring carrier.

In the present invention, the term “pharmaceutically acceptable” means that the composition exhibits the property of being non-toxic to cells or humans exposed to the composition.

More specifically, the pharmaceutical composition may be formulated and used in the form of oral preparations such as powders, granules, tablets, capsules, suspensions, emulsions, syrups, and aerosols, external preparations, suppositories, and sterile injectable solutions according to conventional methods, but is not limited thereto as long as it is a formulation used for the treatment of hair loss in the art.

Specific examples of the carriers, excipients, and diluents that may be contained in the pharmaceutical composition include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, polycaprolactone (PCL), polylactic acid (PLA), poly-L-lactic acid (PLLA), and mineral oil.

In a case where the pharmaceutical composition is formulated into a preparation, the preparation may be prepared using diluents or excipients such as fillers, bulking agents, binders, wetting agents, disintegrants, and surfactants that are commonly used.

Solid preparations for oral administration include tablets, pills, powders, granules, and capsules, and these solid preparations may be prepared by mixing the extract and its fractions with at least one or more excipients, for example, starch, calcium carbonate, sucrose or lactose, and gelatin. In addition to simple excipients, lubricants such as magnesium stearate and talc may also be used.

Liquid preparations for oral administration include suspensions, oral liquids, emulsions, and syrups, and may contain various excipients, such as wetting agents, sweeteners, flavoring agents, and preservatives in addition to the commonly used simple diluents, such as water and liquid paraffin. Preparations for parenteral administration may include sterile aqueous solutions, non-aqueous solutions, suspensions, emulsions, lyophilized preparations, and suppositories. As non-aqueous solvents and suspending agents, propylene glycol, polyethylene glycol, vegetable oils such as olive oil, injectable esters such as ethyl oleate, and the like may be used.

Still another aspect of the present invention provides a method for preventing or treating hair loss, which includes administering a pharmaceutical composition containing the immune-tolerized fetal stem cell-derived extracellular vesicles for hair loss treatment.

The terms alopecia, immune-tolerized fetal stem cell-derived extracellular vesicles, and treatment are as described above.

The term “prevention” of the present invention means any action that suppresses a hair loss disease or delays the onset by the administration of the composition. For the purpose of the present invention, the prevention may be understood as an action that suppresses or delays the onset of a hair loss disease by using the pharmaceutical composition of the present invention, but is not particularly limited thereto.

The term “improvement” of the present invention means any action that at least reduces the degree of hair loss disease.

The term “administration” in the present invention means introducing or treating the pharmaceutical composition of the present invention in a subject by an appropriate method.

The pharmaceutical composition of the present invention may be administered in a pharmaceutically effective amount, and the pharmaceutically effective amount means an amount that is sufficient to treat a disease with a reasonable benefit/risk ratio applicable to medical treatment and does not cause any side effects, and may be readily determined by those skilled in the art based on factors well known in the medical field. The route and method for administering the pharmaceutical composition of the present invention are not particularly limited, and any route and method of administration may be adopted if the composition can reach the subject to achieve the purpose of the present invention.

The “subject” refers to all animals, including humans, that have developed or are capable of developing a hair loss disease.

Still another aspect of the present invention provides a composition for a cosmetic, a functional food, a quasi-drug, or a medical device for treating, preventing, alleviating or improving hair loss, which contains the immune-tolerized fetal stem cell-derived extracellular vesicles for hair loss treatment.

The term “cosmetic” of the present invention means an article that is applied, rubbed, sprayed, or used in a similar manner on the human body to cleanse and beautify the human body, add charm, brighten the appearance, or maintain or enhance the health of skin and hair, and has a mild effect on the human body.

The term “functional food” of the present invention is the same as food for special health use (FoSHU), and refers to a food with high medical and therapeutic effects that is processed to efficiently exhibit a bioregulatory function in addition to nutritional supply. In the present invention, the functional food is used together with a health food or a health supplement.

The term “quasi-drug” of the present invention refers to articles that have a milder effect than drugs among articles that are used for the purpose of diagnosing, treating, improving, alleviating, curing, or preventing diseases in humans or animals. For example, quasi-drugs under the Pharmaceutical Affairs Act are products other than articles used for pharmaceutical purposes, and include products used to treat or prevent diseases in humans/animals and products that have a mild effect on the human body or do not have a direct effect.

The term “medical device” of the present invention refers to an instrument, machine, device, material, or similar product used alone or in combination for humans or animals, and may include products used (i) for the purpose of diagnosing, treating, alleviating, curing or preventing diseases; (ii) for the purpose of diagnosing, treating, alleviating or correcting injuries and disabilities; and (iii) for the purpose of examining, replacing or modifying structures or functions, but excludes drugs and quasi-drugs under the Pharmaceutical Affairs Act.

Still another aspect of the present invention provides the use of the immune-tolerized fetal stem cell-derived extracellular vesicles for hair loss treatment as a composition for a pharmaceutical product, a cosmetic, a functional food, a quasi-drug or a medical device for treating, preventing, alleviating or improving hair loss.

### Example 1

Obtaining the Extracellular Vesicles Through the Co-Culture of Human Dermal Fibroblasts and Adipose-Derived Mesenchymal Stem Cells (HDF/AD-MSCCO-EVs) Containing Extracellular Matrix that Promotes Hair Follicle Formation and Development

In order to simulate the microenvironment that induces the formation and development of fetal hair follicles, a co-culture system of human dermal fibroblasts and adipose-derived mesenchymal stem cells was developed to obtain extracellular vesicles containing extracellular matrix secreted from dermal condensate (DC) that forms hair follicles and preadipocytes that promote differentiation of hair follicle precursor cells into hair follicle cells.

More specifically, in order to exclude foreign antigens that might be contained in the extracellular vesicles, human dermal fibroblasts (PromoCell, Cat. #C-12352) and human adipose-derived mesenchymal stem cells (PromoCell, Cat. #C-12977) were stored in a cryopreservation agent (MBTC-CRYOGUARD®, Stemmedicare) that does not contain fetal bovine serum (FBS) and dimethyl sulfoxide (DMSO) at −80° C. in an ultra-low temperature freezer for 2 weeks.

After each of the stored cells was thawed, adipose-derived stem cells were dispensed into serum-free DMEM/F-12 medium in the lower plate of a multi-plate dish (ThermoFisher, Cat. #140663) at a density of 20,000 cells/cm2, human dermal fibroblasts were dispensed into serum-free DMEM/F-12 medium in the upper insert at a density of 10,000 cells/cm2, respectively, and the co-culture was performed for 120 hours in an incubator at 37° C. and 5% CO2.

Then, the obtained culture supernatant was centrifuged to remove cells and impurities, and vacuum filtrations were performed two times using 0.8 μm and 0.45 μm filters, respectively, to obtain human dermal fibroblasts and adipose-derived mesenchymal stem cells co-cultured extracellular vesicles (HDF/AD-MSCCO-EVs).

In order to compare the content of the extracellular matrix contained in the extracellular vesicles (HDF/AD-MSCCO-EVs) obtained through the co-culture of human dermal fibroblasts and adipose-derived mesenchymal stem cells with those in the extracellular vesicles (HDF-EVs and AD-MSC-EVs) obtained by culturing human dermal fibroblasts and human adipose-derived mesenchymal stem cells, respectively, analysis was performed by LC-MS/MS, and as a result, it was found that collagen types IV, VI, and VII, laminins, and integrins, which are extracellular matrix components that induce the secretion of factors necessary for hair follicle formation and development, hair follicle stem cell activation, and hair cycle transition from the catagen phase to the anagen phase, were contained at much higher levels in HDF/AD-MSCCO-EVs than in HDF-EVs and AD-MSC-EVs (Table 1).

TABLE 1

| Extracellular matrix | HDF-EVs | AD-MSC-EVs | HDF/AD-MSC^{CO}-EVs |
|---|---|---|---|
| proteins | (ng/ml) | (ng/ml) | (ng/ml) |
| Collagen IV | 620 | 97 | 3,478 |
| Collagen VI | 373 | 27 | 892 |
| Collagen VII | 168 | 42 | 1,876 |
| Laminins | 5.4 | 0.8 | 36 |
| Integrins | 7.1 | 1.9 | 69 |

<Comparison of Concentrations of Extracellular Matrix Proteins Contained in Extracellular Vesicles (HDF/AD-MSC^{CO}-EVs) Obtained Through the Co-Culture of Human Dermal Fibroblasts and Adipose-Derived Mesenchymal Stem Cells and Extracellular Vesicles (HDF-EVs and AD-MSC-EVs) Obtained by Culturing Human Dermal Fibroblasts and Human Adipose-Derived Mesenchymal Stem Cells, Respectively>

### Example 2

Manufacturing an Ex Vivo Culture Matrix (MBTC-Matrix®) to Induce Immune Tolerance Properties that can Express and Secrete HLA-G Protein in a Target Cell

An ex vivo culture matrix (MBTC-Matrix®) capable of inducing immune tolerance properties that can express and secrete HLA-G protein in a target cell was manufactured.

More specifically, in order to exclude foreign antigens that might be contained in extracellular vesicles, human amniotic membrane-derived mesenchymal stem cells (ScienCell, Cat. #7140) and mesenchymal stem cells established from amniotic fluid donated from healthy mothers were stored in a cryopreservation agent (MBTC-CRYOGUARD®, Stemmedicare) that does not contain fetal bovine serum (FBS) and dimethyl sulfoxide (DMSO) in at −80° C. in an ultra-low temperature freezer for 2 weeks.

After each of the stored cells was thawed, amniotic membrane-derived mesenchymal stem cells were dispensed into serum-free DMEM/F-12 medium in the lower plate of a multi-plate dish (ThermoFisher, Cat. #140663) and amniotic fluid-derived mesenchymal stem cells were dispensed into serum-free DMEM/F-12 medium in the upper insert at a density of 20,000 cells/cm2, respectively, and the co-culture was performed for 96 hours in an incubator at 37° C. and 5% CO2.

Then, the obtained culture supernatant was centrifuged to remove cells and impurities, and vacuum filtrations were performed two times using 0.8 μm and 0.45 μm filters, respectively, to obtain human amniotic fluid and amniotic membrane-derived mesenchymal stem cell co-cultured extracellular vesicles (AF/AM-MSCCO-EVs).

The ex vivo culture gel was manufactured by adding hyaluronic acid powder to the human amniotic fluid and amniotic membrane-derived mesenchymal stem cell co-cultured extracellular vesicles obtained by the method above so that the pH was 6.6 to 6.8, and mixing it thoroughly by vortexing. The gel was dispensed onto a culture plate and evenly spread by shaking the culture plate on a vibrator, into which human trophoblasts were dispensed, and subculture was performed in serum-free DMEM/F-12K medium at 5 to 6 day intervals.

At this time, to induce progesterone hormone secretion from trophoblasts, a temperature change condition having a 5-day cycle in the range of 36.0° C. to 37.0° C., similar to the body temperature change of women before and after ovulation, and a vibration culture condition having a 24-hour cycle in which the vibration changes in the range of 0 RPM to 60 RPM (excluding 0 RPM) were applied.

In order to examine whether trophoblasts cultured in the ex vivo culture matrix gel could continuously secrete HLA-G protein, while the subculture was performed, the concentration of soluble HLA-G (sHLA-G) protein in the culture supernatant obtained during each subculture was measured with a Human HLA-G ELISA kit (LSBio) using MEM-G/9 antibody.

At this time, when the sHLA-G concentration in the culture supernatant exceeded 20 μg/ml for three consecutive passages or more, immune-tolerized trophoblasts (itTBCs) were considered established, and the subculture was stopped.

The immune-tolerized trophoblasts (itTBCs) established in the ex vivo culture gel were dispensed at a density of 15,000 cells/cm2 into serum-free DMEM/F-12K medium using a general culture plate, and cultured for 96 hours in an incubator at 37° C. and 5% CO2.

Then, the obtained culture supernatant was centrifuged to remove cells and impurities, and vacuum filtrations were performed two times using 0.8 μm and 0.45 μm filters, respectively, to obtain immune-tolerized trophoblast-derived extracellular vesicles (itTBC-EVs), which contained HLA-G protein and progesterone hormone and could induce immune tolerance properties in a target cell.

The itTBC-EVs obtained through this process and the AF/AM-MSCCO-EVs were mixed at a ratio of 1:1 to 2:1, hyaluronic acid powder was added so that the pH was 6.6 to 6.8, and the mixture was thoroughly mixed by vortexing to manufacture an ex vivo culture matrix (MBTC-Matrix®) capable of inducing immune tolerance properties that can express and secrete HLA-G protein in a target cell.

### Example 3

Manufacturing Immune-Tolerized Fetal Stem Cell-Derived Extracellular Vesicles for Hair Loss Treatment with the Efficacy of Promoting Angiogenesis and Normalizing the Hair Cycle

It was attempted to manufacture the immune-tolerized fetal stem cell-derived extracellular vesicles for hair loss treatment with the efficacy of promoting angiogenesis and normalizing the hair cycle according to the present invention.

More specifically, in order to induce secretion of extracellular vesicles containing various components that have the efficacy of promoting hair follicle formation and development, hair follicle stem cell activation, hair cycle transition from the catagen phase to the anagen phase, and angiogenesis, in target stem cells, fetal stem cells (hFSCs) were inoculated into the ex vivo culture matrix (MBTC-Matrix®) to induce immune tolerance properties, which was manufactured in Example 2, and cultured in serum-free DMEM/F-12 medium containing the human dermal fibroblasts and adipose-derived mesenchymal stem cells co-cultured extracellular vesicles (HDF/AD-MSCCO-EVs) of Example 1 at 10% v/v. In order to induce immune tolerance properties in fetal stem cells, the cells were subcultured in an incubator at 37° C. and 5% CO2 by applying a temperature change condition having a 5-day cycle and a vibration condition the same as those in Example 2.

In order to examine whether fetal stem cells cultured in the ex vivo culture matrix (MBTC-Matrix®) that induces immune tolerance properties could continuously secrete HLA-G protein, while the subculture was performed, the concentration of soluble HLA-G (sHLA-G) protein in the culture supernatant obtained during each subculture was measured using a Human HLA-G ELISA kit (LSBio) using MEM-G/9 antibody. When the sHLA-G concentration in the culture supernatant exceeded 20 μg/ml for three consecutive passages or more, immune-tolerized fetal stem cells (itHT-FSCs) for producing extracellular vesicles for hair loss treatment were considered established, and the subculture was stopped.

The itHT-FSCs established in the ex vivo culture matrix (MBTC-Matrix®) as described above were washed and inoculated onto a general cell culture plate at a density of 2.0×104 cells/cm2, serum-free DMEM/F-12 medium was added, and the cells were cultured for 96 hours in an incubator at 37° C. and 5% CO2.

The collected culture supernatant was centrifuged to remove cells and impurities, and vacuum filtrations were performed two times using 0.8 μm and 0.45 μm filters, respectively, and tangential flow filtration (TFF) through a 10 kD hollow fiber filter was performed to obtain immune-tolerized fetal stem cell-derived extracellular vesicles for hair loss treatment (itHT-FSC-EVs).

### Example 4

Characterization of Immune-Tolerized Fetal Stem Cell-Derived Extracellular Vesicles for Hair Loss Treatment (itHT-FSC-EVs) with the Efficacy of Promoting Angiogenesis and Normalizing the Hair Cycle

Characterization of the immune-tolerized fetal stem cell-derived extracellular vesicles for hair loss treatment (itHT-FSC-EVs) with the efficacy of promoting angiogenesis and normalizing the hair cycle of the present invention was performed.

### 1) Nanoparticle Tracking Analysis (NTA)

The size and concentration of extracellular vesicles were analyzed using the NS300 (Malvern Panalytical), a nanoparticle tracking analysis (NTA) equipment that could quantitatively measure the particle number concentration and size of extracellular vesicles.

In order to evaluate the concentration efficiency of extracellular vesicles by tangential flow filtration (TFF), a high-concentration process, during the manufacturing process of the immune-tolerized fetal stem cell-derived extracellular vesicles for hair loss treatment (itHT-FSC-EVs) of the present invention, NTA was performed on the extracellular vesicles before and after TFF. In order to match the concentration of the analytical sample to the optimal analytical concentration (1.0×107 to 1.0×109 particles/ml) of extracellular vesicles according to the equipment manufacturer's recommendation, NTA was performed on extracellular vesicle samples diluted 10- to 100-fold.

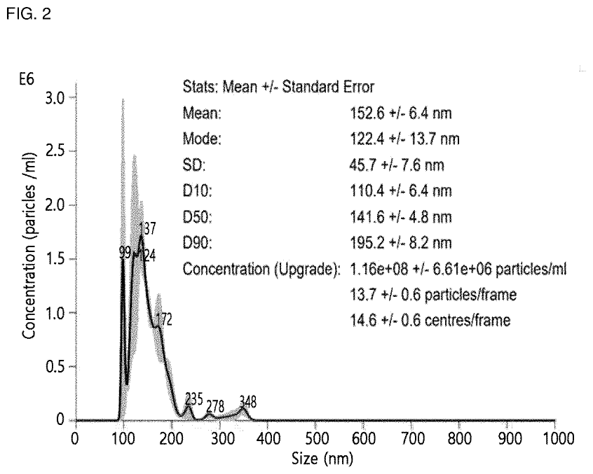

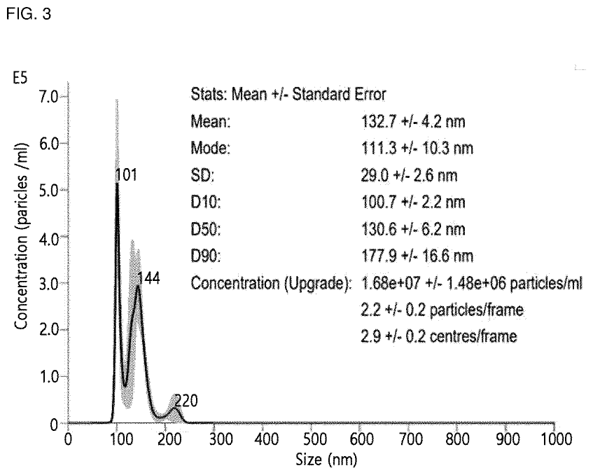

The extracellular vesicles were diluted 10-fold (FIG. 2) and 100-fold (FIG. 3) before TFF, and NTA was performed, and as a result, the average size measured was 152.6 nm and 132.7 nm, respectively, the average particle number concentration measured was 1.16×108 and 1.68×107 particles/ml, respectively, and the average particle number concentration reflecting the dilution factor was 1.16 to 1.68×109 particles/ml.

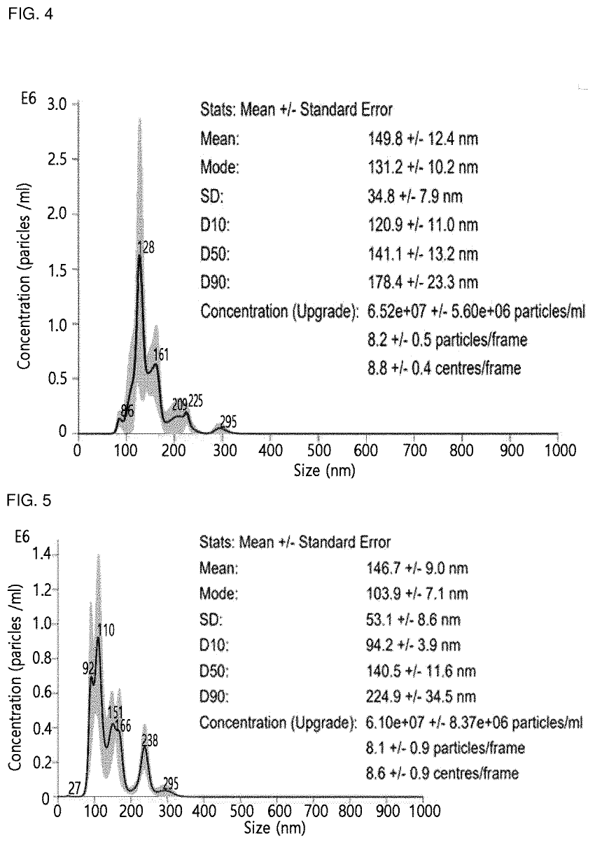

Two lots of extracellular vesicles, each manufactured independently, were diluted 100-fold after TFF, and NTA was performed, and as a result, the average size measured was 149.8 nm and 146.7 nm, respectively, the average particle number concentration measured was 6.52×107 and 6.10×107 particles/ml, respectively, and the average particle number concentration reflecting the dilution factor was 6.10 to 6.52×109 particles/ml, and it was found that extracellular vesicles could be concentrated by about 5-fold through the TFF process (FIGS. 4 and 5).

It was found that the average size and average particle number concentration of extracellular vesicles concentrated by TFF showed a distribution within a reproducible range compared to extracellular vesicles before TFF. The extracellular vesicles concentrated by TFF were diluted 100-fold considering the characteristics of the NTA equipment, and NTA was performed, and as a result, it was found that the average size and average particle number concentration showed a distribution within a reproducible range for each lot.

### 2) Analysis the Contents of Proteins and Genes Related to Promoting Angiogenesis

The contents of proteins and genes, which are related to angiogenesis promotion, contained in the immune-tolerized fetal stem cell-derived extracellular vesicles for hair loss treatment (itHT-FSC-EVs) of the present invention were analyzed.

As controls, (i) immune-tolerized fetal stem cell-derived extracellular vesicles (itFSC-EVs) cultured in an ex vivo culture matrix (MBTC-Matrix®) not containing the human dermal fibroblasts and adipose-derived mesenchymal stem cells co-cultured extracellular vesicles (HDF/AD-MSCCO-EVs) of Example 1 and (ii) human adipose mesenchymal stem cell-derived extracellular vesicles (AD-MSC-EVs) cultured in a general culture plate were set, and the respective extracellular vesicles were obtained from the same number of stem cells under the same culture conditions by the same manufacturing process.

First, the contents of proteins, which are related to angiogenesis promotion, contained in each of the extracellular vesicles were measured three times using the Human Angiogenesis Array Q1 (Raybiotech, Catalog #. QAH-ANG-1-1), and the average concentration was calculated.

As a result, it was found that the contents of all proteins, including EGF and bFGF, which were not detected in AD-MSC-EVs among the proteins related to angiogenesis promotion, such as angiogenin, angiopoietin-2, epidermal growth factor (EGF), basic fibroblast growth factor (bFGF), heparin-binding EGF-like growth factor (HB-EGF), hepatocyte growth factor (HGF), platelet-derived growth factor BB (PDGF-BB), placenta growth factor (PIGF), and vascular endothelial growth factor (VEGF), were at much higher levels in itFSC-EVs and itHT-FSC-EVs, which are fetal stem cell-derived extracellular vesicles, compared to AD-MSC-EVs.

In particular, it was found that the contents of all proteins related to angiogenesis promotion were at higher levels in itHT-FSC-EVs than in itFSC-EVs, and the contents of bFGF and VEGF among these were significantly increased in itHT-FSC-EVs of the present invention compared to itFSC-EVs (Table 2).

TABLE 2

| | itHT-FSC-EVs | itFSC-EVs | AD-MSC-EVs |
|---|---|---|---|
| Proteins | (pg/ml) | (pg/ml) | (pg/ml) |
| Angiogenin | 6,524 | 5,887 | 1,468 |
| Angiopoietin-2 | 9,257 | 8,097 | 2,494 |
| EGF | 123.8 | 90.7 | 0.0 |
| bFGF (FGF-2) | 190.8 | 84.3 | 0.0 |
| HB-EGF | 90.3 | 82.8 | 20.4 |
| HGF | 2,873.3 | 2,141.4 | 268.3 |
| PDGF-BB | 815.0 | 598.1 | 32.0 |
| PIGF | 467.6 | 312.8 | 13.8 |
| VEGF | 10,840.5 | 6,576.4 | 590.7 |

<Analysis and Comparison of Contents of Proteins Related to Angiogenesis Promotion Contained in itHT-FSC-EVs, itFSC-EVs, and AD-MSC-EVs>

The expression patterns of genes, which are related to angiogenesis promotion, contained in the respective extracellular vesicles were compared through exosomal RNA analysis using next generation sequencing (NGS).

As a result, it was found that the expression levels of genes that promote angiogenesis, such as EGFL7, RAMP1, GATA2, CCL2, and RASIP1, were all higher in itHT-FSC-EVs and itFSC-EVs, which are fetal stem cell-derived extracellular vesicles, compared to AD-MSC-EVs.

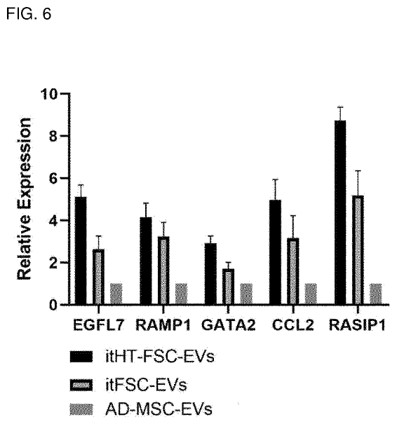

In particular, it was found that each gene was expressed at a similar or higher level in itHT-FSC-EVs of the present invention compared to itFSC-EVs (FIG. 6).

3) Expression Analysis of Proteins and Genes that Normalize the Hair Cycle and Induce the Transition to the Anagen Phase

The contents of proteins and genes, which normalize the hair cycle by promoting hair follicle formation and development and inducing the transition to the anagen phase, contained in the immune-tolerized fetal stem cell-derived extracellular vesicles for hair loss treatment (itHT-FSC-EVs) of the present invention were analyzed.

As controls, (i) immune-tolerized fetal stem cell-derived extracellular vesicles (itFSC-EVs) cultured in an ex vivo culture matrix (MBTC-Matrix®) not containing the human dermal fibroblasts and adipose-derived mesenchymal stem cells co-cultured extracellular vesicles (HDF/AD-MSCCO-EVs) of Example 1 and (ii) human adipose mesenchymal stem cell-derived extracellular vesicles (AD-MSC-EVs) cultured in a general culture plate were set, and the respective extracellular vesicles were obtained from the same number of stem cells under the same culture conditions by the same manufacturing process.

First, the contents of proteins, which are related to hair cycle normalization, contained in each of the extracellular vesicles were measured three times using the Human Growth Factor Array Q1 (Raybiotech, Catalog #. QAH-GF-1-1), and the average concentration was calculated.

As a result, it was found that proteins such as FGF-7 and PDGF-α, which normalized hair cycle by inducing conversion into hair in the anagen phase were contained at much higher levels in itFSC-EVs and itHT-FSC-EVs, which were fetal stem cell-derived extracellular vesicles, compared to AD-MSC-EVs.

In particular, it was found that each protein was contained at a higher level in itHT-FSC-EVs of the present invention than in itFSC-EVs (Table 3).

TABLE 3

| | itHT-FSC-EVs | itFSC-EVs | AD-MSC-EVs |
|---|---|---|---|
| Proteins | (pg/ml) | (pg/ml) | (pg/ml) |
| FGF-7 (KGF) | 739.6 | 594.8 | 49.3 |
| PDGF-A | 815.0 | 463.3 | 32.0 |

<Analysis an Comparison of Contents of Proteins, which are Related to Hair Cycle Normalization, Contained in itHT-FSC-EVs, itFSC-EVs, and AD-MSC-EVs>

The expression patterns of genes, which are related to hair cycle normalization, contained in each of the extracellular vesicles were compared through exosomal RNA analysis using next generation sequencing (NGS).

As a result, it was found that the expression levels of genes that normalize the hair cycle by inducing hair follicle formation and development and the transition to the anagen phase, such as Noggin and SOSTDC1, were higher in itHT-FSC-EVs and itFSC-EVs, which are fetal stem cell-derived extracellular vesicles, compared to AD-MSC-EVs.

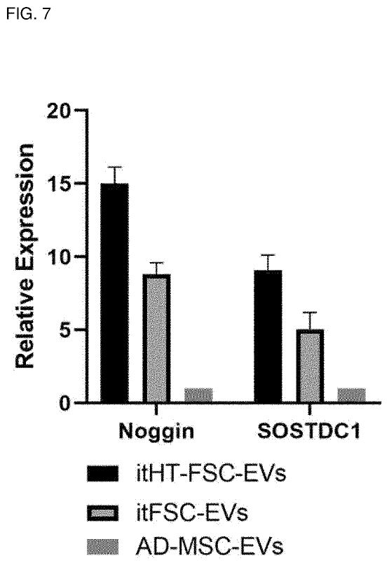

In particular, it was found that each gene was expressed at a similar or higher level in itHT-FSC-EVs of the present invention compared to itFSC-EVs (FIG. 7).

4) Expression Analysis of miRNA Suppressing the DKK1 Gene Expression

The expression patterns of miRNA components, which suppress DKK1 gene expression, contained in the immune-tolerized fetal stem cell-derived extracellular vesicles for hair loss treatment (itHT-FSC-EVs) of the present invention were compared through exosomal miRNA analysis using next generation sequencing (NGS).

As controls, (i) immune-tolerized fetal stem cell-derived extracellular vesicles (itFSC-EVs) cultured in an ex vivo culture matrix (MBTC-Matrix®) not containing the human dermal fibroblasts and adipose-derived mesenchymal stem cells co-cultured extracellular vesicles (HDF/AD-MSCCO-EVs) of Example 1 and (ii) human adipose mesenchymal stem cell-derived extracellular vesicles (AD-MSC-EVs) cultured in a general culture plate were set, and the respective extracellular vesicles were obtained from the same number of stem cells under the same culture conditions by the same manufacturing process.

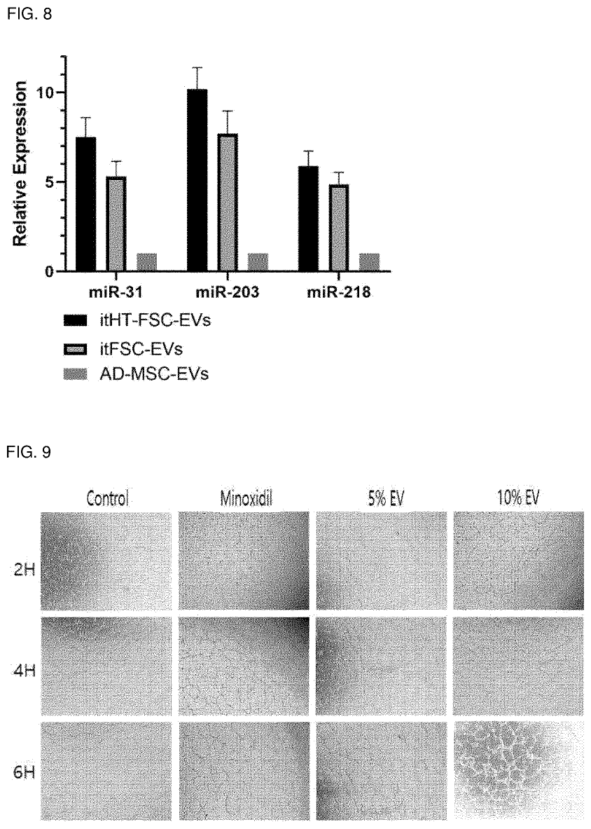

As a result, it was found that the expression levels of various microRNAs (miR-31, miR-203, and miR-218) that suppress DKK1 gene expression were higher in itHT-FSC-EVs and itFSC-EVs, which are fetal stem cell-derived extracellular vesicles, compared to AD-MSC-EVs. In particular, it was found that each miRNA was expressed at a higher level in itHT-FSC-EVs of the present invention compared to itFSC-EVs (FIG. 8).

### Example 5

Examination of Angiogenesis Promoting Efficacy of Immune-Tolerized Fetal Stem Cell-Derived Extracellular Vesicles for Hair Loss Treatment (itHT-FSC-EVs) with the Efficacy of Promoting Angiogenesis and Normalizing the Hair Cycle

The angiogenesis promoting efficacy of the immune-tolerized fetal stem cell-derived extracellular vesicles for hair loss treatment (itHT-FSC-EVs) of the present invention was evaluated using human umbilical vein endothelial cells (HUVECs).

More specifically, HUVECs (Promocell, Cat. #C-12200) were cultured in a dedicated culture medium (Promocell, Cat. #C-22010) and then inoculated at a density of 1×105 cells/well in a 24-well plate coated with Matrigel (Corning, Cat. #354234), and then the immune-tolerized fetal stem cell-derived extracellular vesicles for hair loss treatment (itHT-FSC-EVs) of the present invention were added at 5% (50 μl/ml) and 10% (100 μl/ml), respectively. As a positive control, 10 μM minoxidil (Sigma-Aldrich, Cat. #1444208) was added.

Microscopic images of HUVECs taken 2 and 6 hours after the addition of each substance were compared, and as a result, more angiogenesis was observed in the itHT-FSC-EVs treatment groups of the present invention compared to the minoxidil treatment group, and it was found that the angiogenesis efficacy increased in a concentration-dependent manner (FIG. 9).

In order to quantify the angiogenesis promoting efficacy of the immune-tolerized fetal stem cell-derived extracellular vesicles for hair loss treatment (itHT-FSC-EVs) of the present invention, the microscopic images in FIG. 9 were analyzed using ImageJ software. For detailed analysis methods, refer to the literature ^{┌}Angiogenesis Analyzer for ImageJ—A comparative morphometric analysis of “Endothelial Tube Formation Assay” and “Fibrin Bead Assay”_{┘} (Scientific Reports, 2020).

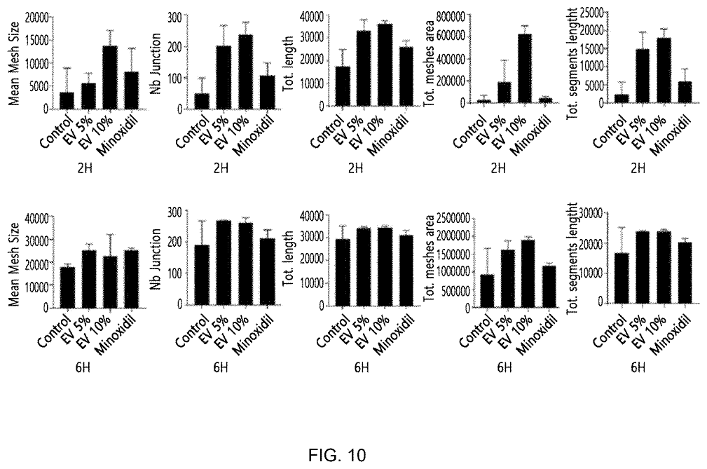

As a result, it was found that indicators such as the mean mesh size (Mean Mesh Size), number of junctions per same-area image (Nb Junction), total length of all branches and segments per same-area image (Tot. length), total meshes area per same-area image (Tot. meshes area), and total segments length per same-area image (Tot. segments length), which are used to evaluate the angiogenesis promoting efficacy, increased in a concentration-dependent manner in the itHT-FSC-EVs treatment groups of the present invention compared to the minoxidil treatment group (FIG. 10).

From the above results, it was found that the immune-tolerized fetal stem cell-derived extracellular vesicles for hair loss treatment (itHT-FSC-EVs) of the present invention have superior angiogenesis promoting efficacy on HUVECs compared to minoxidil.

### Example 6

Examination of Hair Cycle Normalizing Efficacy of Immune-Tolerized Fetal Stem Cell-Derived Extracellular Vesicles for Hair Loss Treatment (itHT-FSC-EVs) with the Efficacy of Promoting Angiogenesis and Normalizing the Hair Cycle

The hair cycle normalizing efficacy of the immune-tolerized fetal stem cell-derived extracellular vesicles for hair loss treatment (itHT-FSC-EVs) of the present invention was evaluated using human follicle dermal papilla cells (HFDPCs).

Specifically, HFDPCs (Promocell, Cat. #C-12071) were inoculated at a density of 4×105 cells/well in a 24-well plate, then 100 ng/ml of DKK1 (Peprotech, Cat. #120-30) was added to the dedicated culture medium (Promocell, Cat. #C-26501), excluding the control group, and the culture was performed for 3 days to establish a catagen-induced model.

The immune-tolerized fetal stem cell-derived extracellular vesicles for hair loss treatment (itHT-FSC-EVs) of the present invention were added to the catagen HFDPCs induced by DKK1 at 5% (50 μl/ml) and 10% (100 μl/ml), respectively. As positive control groups, 10 μM minoxidil (Sigma-Aldrich, Cat. #1444208) and 100 ng/ml FGF-12 (Elabscience, Cat. #PKSH032433), which are known to induce the anagen phase by Woo et al. (Int. J. Mol. Sci., 2022, 23(16)), were added, respectively.

HFDPCs were treated with each substance, and a sufficient amount of RNA (100 ng/μl or more) was obtained from the HFDPCs 2 days later using Nano-drop (ND 2000), and then the expression patterns of genes that maintain the catagen phase and genes that promote the transition to the anagen phase were compared through NGS, respectively.

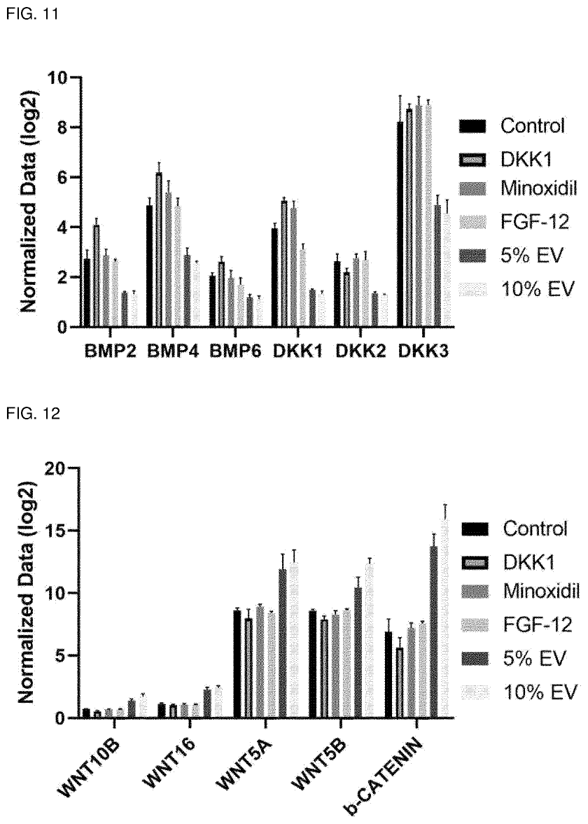

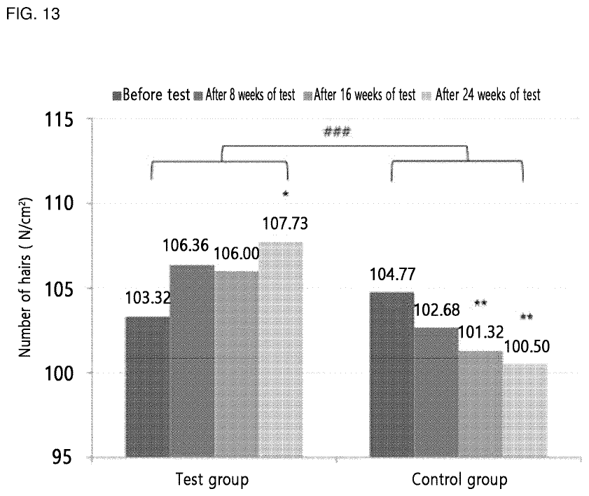

First, it was found that the gene expression of each of BMP2, BMP4, and BMP6 among major BMPs that maintain the catagen phase and inhibit transition to the anagen phase in HFDPCs and HFSCs, and DKK1, DKK2, and DKK3 that inhibit the Wnt/β-catenin signaling, a key signaling pathway for hair growth and differentiation, increased in the DKK1 treatment groups compared to the control group. It was found that the expression of these genes was slightly reduced in the minoxidil and FGF-12 treatment groups as positive control groups, but the expression of genes that maintain the catagen phase and inhibit transition to the anagen phase was significantly reduced in a concentration-dependent manner in the group treated with the immune-tolerized fetal stem cell-derived extracellular vesicles for hair loss treatment (itHT-FSC-EVs) of the present invention (FIG. 11).

It was found that the gene expression of each of WNT10B, WNT16, WNT5A, WNT5B, and β-catenin, which promote transition to the anagen phase in HFDPCs and play a role in key signaling in hair growth and differentiation, was reduced in the DKK1 treatment groups compared to the control group. It was found that the expression of these genes increased slightly or did not significantly change in the minoxidil and FGF-12 treatment groups as positive control groups, but the expression of genes, which promote transition to the anagen phase and play a role in key signaling in hair growth and differentiation, was significantly increased in a concentration-dependent manner in the group treated with the immune-tolerized fetal stem cell-derived extracellular vesicles for hair loss treatment (itHT-FSC-EVs) of the present invention (FIG. 12).

From the above results, it was found that the immune-tolerized fetal stem cell-derived extracellular vesicles for hair loss treatment (itHT-FSC-EVs) of the present invention have superior hair cycle normalizing efficacy on HFDPCs compared to minoxidil and FGF-12.

### Example 7

Evaluation of Hair Loss Symptom Alleviating Efficacy of Immune-Tolerized Fetal Stem Cell-Derived Extracellular Vesicles for Hair Loss Treatment (itHT-FSC-EVs) with the Efficacy of Promoting Angiogenesis and Normalizing the Hair Cycle

In order to evaluate the efficacy of normalizing the hair cycle and alleviating hair loss symptoms of the immune-tolerized fetal stem cell-derived extracellular vesicles for hair loss treatment (itHT-FSC-EVs) of the present invention, a human application test was conducted on subjects diagnosed with alopecia.

An external preparation (Hair Retro-Genesis Ampoule) containing itHT-FSC-EVs of the present invention was set as a test group for human application test, and an external preparation of the same formulation but not containing itHT-FSC-EVs was set as a control group.

Specifically, after obtaining approval from the Institutional Ethics Committee of the Korean Institute of Dermatological Sciences, a specialized institution for human application testing, each test substance was directly applied to the scalp and hair of 44 research subjects diagnosed with androgenetic alopecia aged 18 to 54 years once a day for 24 weeks and then the number of hairs was analyzed using phototrichogram after 8, 16, and 24 weeks of the test, respectively.

### 1) Evaluation of Effectiveness in Alleviating Hair Loss Symptoms

As a result of measuring the number of hairs, in the test group, the number of hairs was 106.36±19.07/cm2 at 8 weeks, 106.00±20.06/cm2 at 16 weeks, and 107.73±17.56/cm2 at 24 weeks and increased to a statistically significant level at 24 weeks compared to 103.32±19.22/cm2 before the test. However, in the control group, the number of hairs was 102.68±12.75/cm2 at 8 weeks, 101.32±14.09/cm2 at 16 weeks, and 100.50±11.07/cm2 at 24 weeks and decreased to a statistically significant level after 16 weeks compared to 104.77±12.85/cm2 before the test (FIG. 13).

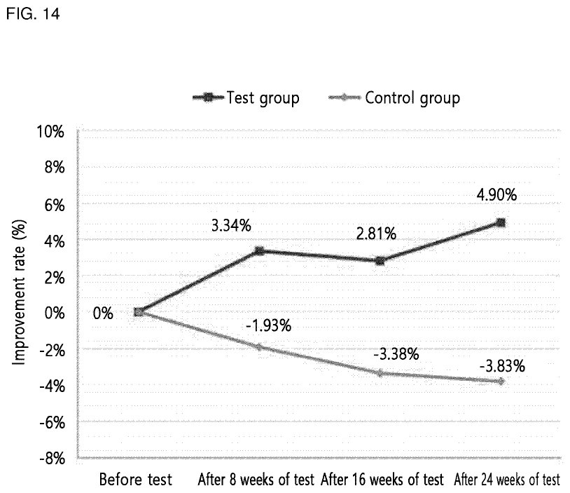

As result of analyzing the improvement rate of the number of hairs, the test group had an improvement rate of 3.34% at 8 weeks, 2.81% at 16 weeks, and 4.90% at 24 weeks compared to the number of hairs before the test, and the number of hairs improved to a statistically significant level at 24 weeks. However, in the control group, the change rate was −1.93% at 8 weeks, −3.38% at 16 weeks, and −3.83% at 24 weeks compared to the number of hairs before the test, and improvement to a statistically significant level was not found at any week (FIG. 14).

### 2) Evaluation of Efficacy of Improving the Number of Lost Hairs

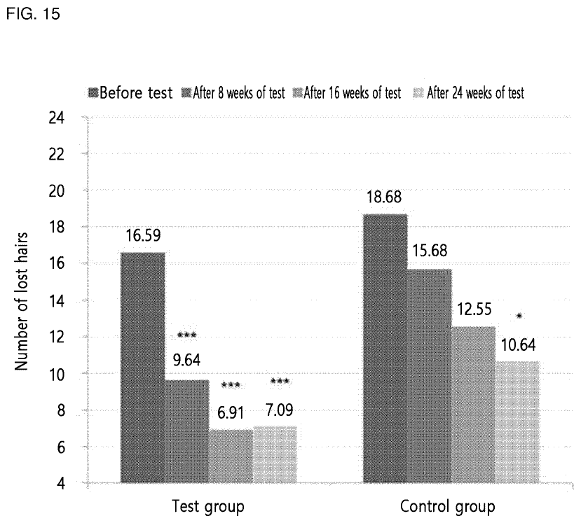

As a result of measuring the number of lost hairs, in the test group, the number of lost hairs was 9.64±13.40 at 8 weeks, 6.91±6.78 at 16 weeks, and 7.09±7.00 at 24 weeks, and decreased to a statistically significant level after 8 weeks of the test compared to 16.59±13.48 before the test. However, in the control group, the number of lost hairs was 15.68±13.50 at 8 weeks, 12.55±9.86 at 16 weeks, and 10.64±7.58 at 24 weeks and decreased to a statistically significant level only at 24 weeks compared to 18.68±12.18 before the test (FIG. 15).

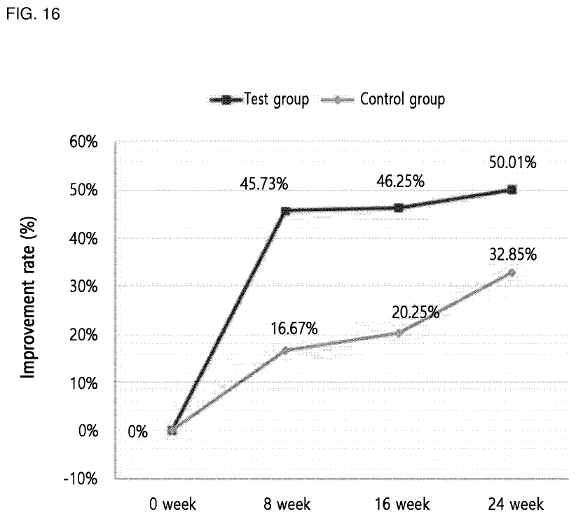

As a result of analyzing the improvement rate of the number of lost hairs, the test group had an improvement rate of 45.73% at 8 weeks, 46.251% at 16 weeks, and 50.01% at 24 weeks compared to the number of lost hairs before the test, and the number of lost hairs was improved to a statistically significant level after 8 weeks. However, in the control group, the improvement rate was 16.67% at 8 weeks, 20.25% at 16 weeks, and 32.85% at 24 weeks compared to the number of lost hairs before the test, and improvement to a statistically significant level was found only at 24 weeks (FIG. 16).

### 3) Evaluation of Hair Cycle Normalizing Efficacy

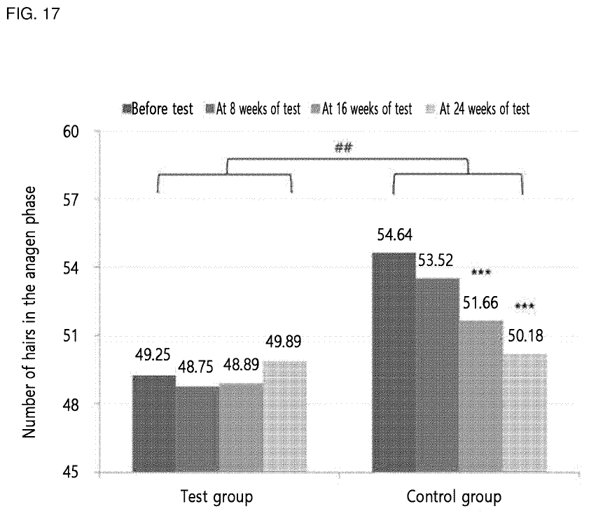

As a result of measuring the number of hairs in the anagen phase, in the test group, the number of hairs in the anagen phase was 48.75±10.51 at 8 weeks, 48.89±10.42 at 16 weeks, and 49.89±10.99 at 24 weeks, and a statistically significant change was not found compared to 49.25±9.82 before the test, but the number increased at 24 weeks. However, in the control group, the number of hairs in the anagen phase was 53.52±10.87 at 8 weeks, 51.66±9.18 at 16 weeks, and 50.18±9.61 at 24 weeks, and decreased to a statistically significant level after 16 weeks compared to 54.64±9.64 before the test (FIG. 17).

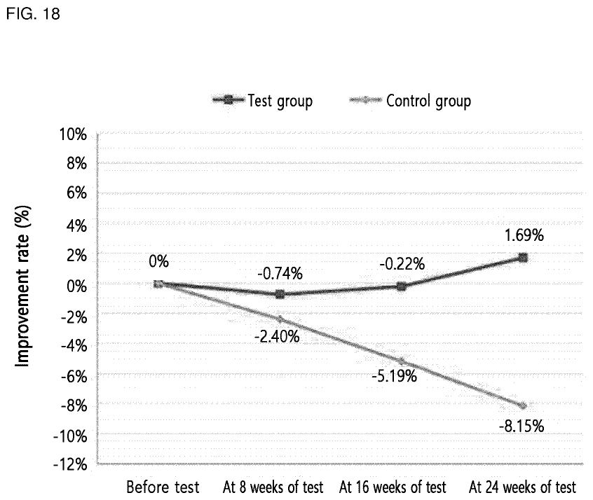

As a result of analyzing the improvement rate of the number of hairs in the anagen phase, the test group had an improvement rate of −0.74% at 8 weeks, −0.22% at 16 weeks, and 1.69% at 24 weeks compared to the number of hairs in the anagen phase before the test. However, in the control group, the improvement rate was −2.40% at 8 weeks, −5.19% at 16 weeks, and −8.155% at 24 weeks compared to the number of hairs in the anagen phase before the test, and the number of hairs in the anagen phase decreased to a statistically significant level after 16 weeks (FIG. 18).

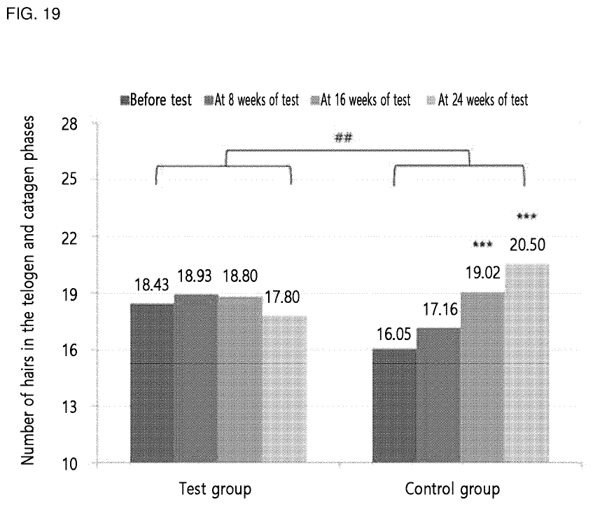

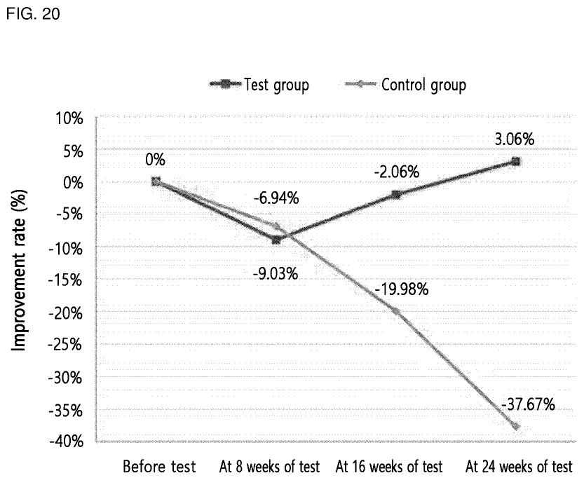

As a result of measuring the number of hairs in the telogen and catagen phases, in the test group, the number of hairs in the telogen and catagen phases was 18.93±7.01 at 8 weeks, 18.80±8.85 at 16 weeks, and 17.80±8.73 at 24 weeks and a statistically significant change was not found compared to 18.43±6.72 before the test, but the number decreased at 24 weeks. However, in the control group, the number of hairs in the telogen and catagen phases was 17.16±8.67 at 8 weeks, 19.02±7.82 at 16 weeks, and 20.50±8.41 at 24 weeks and increased to a statistically significant level after 16 weeks compared to 16.05±7.00 before the test (FIG. 19).

As a result of analyzing the improvement rate of the number of hairs in the telogen and catagen phases, the test group had an improvement rate of −9.03% at 8 weeks, −2.06% at 16 weeks, and 3.06% at 24 weeks compared to the rate before the test. However, in the control group, the improvement rate was −6.94% at 8 weeks, −19.98% at 16 weeks, and −37.67% at 24 weeks compared to the number of hairs in the telogen and catagen phases before the test, and the number of hairs in the telogen and catagen phases increased to a statistically significant level after 16 weeks (FIG. 20).

Through the human application test, it was found that the immune-tolerized fetal stem cell-derived extracellular vesicles for hair loss treatment (itHT-FSC-EVs) of the present invention have the hair cycle normalizing efficacy by maintaining the anagen phase and delaying the entry into the telogen and catagen phases, and have a therapeutic efficacy in alleviating hair loss symptoms in patients with alopecia.

For reference, the present inventor determined that it would be greatly important to manufacture immune-tolerized extracellular vesicles containing human leukocyte antigen G (HLA-G) protein, which establishes an immune tolerant environment that perfectly protects the fetus from the mother's immune system during pregnancy in order for the extracellular vesicles, which are the main component of the composition for hair loss treatment of the present invention, to be completely delivered to hair follicle stem cells and hair follicle cells without causing an immune response due to immunogenicity even when repeatedly administered, and to exhibit hair loss treating efficacy.

To this end, the present inventor established ex vivo culture conditions that simulate the in vivo environment during pregnancy, which can induce the continuous secretion and expression of HLA-G protein, which establishes an immune tolerant environment that protects the fetus from the mother's immune system during pregnancy, through prior domestically registered patents 10-1970726, 10-2049505, and 10-2252325 (U.S. Ser. Nos. 11/566,220 B2, 11/771,720 B2 and 12/150,962 B2), which are incorporated into the present invention by reference in their entirety. Through this, the present inventor induced immune tolerance properties in stem cells for producing extracellular vesicles for hair loss treatment.

The present inventor has newly established an ex vivo culture system for producing extracellular vesicles for hair loss treatment that has the efficacy of normalizing the hair cycle and improving blood circulation, regardless of various causes of hair loss, such as genetic and hormonal factors by applying an ex vivo culture matrix containing various extracellular matrix components secreted from human dermal fibroblasts and adipose-derived stem cells to the established ex vivo culture conditions for inducing immune tolerance properties, particularly in order to obtain extracellular vesicles that contain various factors involved in the formation and development of hair follicles much more abundantly by using fetal stem cells before the 15th week of pregnancy, which is the period when scalp hair follicles are formed.

It was verified that the immune-tolerized fetal stem cell-derived extracellular vesicles for hair loss treatment produced by the newly established ex vivo culture system do not induce immune rejection due to their own immunogenicity, as well as contain miRNA components that suppress the expression of DKK1 gene, which induces hair loss, components that promote hair follicle formation and development, normalize the hair cycle, and induce transition to the anagen phase by inhibiting bone morphogenetic protein (BMP) signaling, which maintains HFSCs in a quiescent state to inhibit differentiation into hair follicle cells and maintains quiescent hair follicles, and activating Wnt/β-catenin signaling, and components that promote the improvement of blood circulation at much higher levels compared to extracellular vesicles produced under general culture conditions and ex vivo culture conditions inducing immune tolerance properties, and have superior efficacy in normalizing the hair cycle and treating hair loss through human efficacy evaluation targeting patients with hair loss, whereby the present invention has been completed.

The embodiments of the present invention described above have been described with reference to embodiments illustrated in the drawings to help understanding, but these are merely exemplary, and those skilled in the art will appreciate that various modifications and equivalent other embodiments are possible from this. Therefore, the true technical protection scope of the present invention should be defined by the appended claims.

1. A method for manufacturing immune-tolerized fetal stem cell-derived extracellular vesicles for hair loss treatment, the method comprising:
(a) obtaining extracellular vesicles containing extracellular matrix that promote hair follicle formation and development through the co-culture of human dermal fibroblasts and adipose-derived mesenchymal stem cells;
(b) preparing an ex vivo culture matrix that induces immune tolerance properties that can continuously express and secrete HLA-G protein in a target cell; and
(c) inoculating fetal stem cells into the ex vivo culture matrix that induces immune tolerance properties and subculturing the cells in a serum-free medium containing the extracellular vesicles obtained through the co-culture of human dermal fibroblasts and adipose-derived mesenchymal stem cells in step (a) under temperature change and vibration culture conditions similar to those in the body during pregnancy, wherein
the temperature change and vibration culture conditions similar to those in the body during pregnancy in step (c) are a temperature change condition having a 5-day cycle in which a temperature changes in a range of 36.0° C. to 37.0° C. and a vibration culture condition having a 24-hour cycle in which a vibration changes in a range of 0 RPM to 60 RPM (excluding 0 RPM).

2. The method for manufacturing immune-tolerized fetal stem cell-derived extracellular vesicles for hair loss treatment according to claim 1, further comprising:
(d) inoculating the subcultured immune-tolerized fetal stem cells into a culture plate, culturing the cells in a serum-free medium, and obtaining a culture supernatant; and
(e) performing multi-stage membrane filtration and tangential flow filtration on the culture supernatant to separate extracellular vesicles at a high concentration.

3. The method for manufacturing immune-tolerized fetal stem cell-derived extracellular vesicles for hair loss treatment according to claim 1, wherein the extracellular vesicles obtained through the co-culture of human dermal fibroblasts and adipose-derived mesenchymal stem cells in step (a) are manufactured through
(a-1) inoculating human dermal fibroblasts and adipose-derived mesenchymal stem cells onto upper and lower parts of a co-culture plate, respectively;
(a-2) performing the co-culture in a serum-free medium and obtaining a culture supernatant; and
(a-3) performing multi-stage filtration and separation on the culture supernatant.

4. The method for manufacturing immune-tolerized fetal stem cell-derived extracellular vesicles for hair loss treatment according to claim 1, wherein the extracellular vesicles obtained through the co-culture of human dermal fibroblasts and adipose-derived mesenchymal stem cells in step (a) contain collagen types IV, VI and VII, laminins and integrins, which are extracellular matrix components that induce secretion of factors necessary for hair follicle formation and development, hair follicle stem cell activation, and hair cycle transition from a catagen phase to an anagen phase, in fetal stem cells.

5. The method for manufacturing immune-tolerized fetal stem cell-derived extracellular vesicles for hair loss treatment according to claim 1, wherein the ex vivo culture matrix having immune tolerance properties that can continuously express and secrete HLA-G protein in a target cell of step (b) is manufactured through
(b-1) obtaining extracellular vesicles through the co-culture of human amniotic membrane-derived mesenchymal stem cells and amniotic fluid-derived mesenchymal stem cells;
(b-2) inoculating human trophoblasts into an ex vivo culture gel containing hyaluronic acid and the extracellular vesicles;
(b-3) culturing the trophoblasts under temperature change and vibration culture conditions similar to those in the body during pregnancy, wherein
the temperature change and vibration culture conditions similar to those in the body during pregnancy are a temperature change condition having a 5-day cycle in which a temperature changes in a range of 36.0° C. to 37.0° C. and a vibration culture condition having a 24-hour cycle in which a vibration changes in a range of 0 RPM to 60 RPM (excluding 0 RPM);
(b-4) obtaining immune-tolerized trophoblast-derived extracellular vesicles through serum-free culture of trophoblasts with induced immune tolerance properties; and
(b-5) adding hyaluronic acid to each of the extracellular vesicles obtained in step (b-1) and the extracellular vesicles obtained in step (b-4) and performing mixing.

6. The method for manufacturing immune-tolerized fetal stem cell-derived extracellular vesicles for hair loss treatment according to claim 1, wherein the ex vivo culture matrix in step (c) maintains an acidic condition of pH 6 to 7.

7. The method for manufacturing immune-tolerized fetal stem cell-derived extracellular vesicles for hair loss treatment according to claim 2, wherein the subcultured fetal stem cells of step (d) have immune tolerance properties that express HLA-G protein on a cell surface or secrete HLA-G protein outside a cell and HLA-G protein is present in a culture supernatant.

8. The method for manufacturing immune-tolerized fetal stem cell-derived extracellular vesicles for hair loss treatment according to claim 1, wherein the obtained fetal stem cell-derived extracellular vesicles reduce gene expression of BMP2, BMP4, BMP6, DKK1, DKK2 and DKK3, which maintain hair catagen phase in human hair follicle dermal papilla cells and induce hair loss, thereby alleviating hair loss symptoms.

9. The method for manufacturing immune-tolerized fetal stem cell-derived extracellular vesicles for hair loss treatment according to claim 1, wherein the obtained fetal stem cell-derived extracellular vesicles increase gene expression of WNT10B, WNT16, WNT5A, WNT5B and β-catenin, which induce an anagen phase in human hair follicle dermal papilla cells and promote hair follicle growth and differentiation, thereby alleviating hair loss symptoms.

10. A pharmaceutical composition for hair loss treatment comprising immune-tolerized fetal stem cell-derived extracellular vesicles that are manufactured by the method according to claim 1, wherein
the extracellular vesicles contain noggin, sclerostin domain-containing protein 1 (SOSTDC1), fibroblast growth factor-7 (FGF-7), and platelet-derived growth factor-alpha (PDGF-α) to promote conversion of hair in the catagen phase into hair in the anagen phase.

11. The pharmaceutical composition for hair loss treatment comprising immune-tolerized fetal stem cell-derived extracellular vesicles according to claim 10, wherein the extracellular vesicles additionally contain vascular endothelial growth factor (VEGF), fibroblast growth factor-2 (FGF-2), epidermal growth factor-like domain-containing protein 7 (EGFL7), receptor activity-modifying protein 1 (RAMP1), GATA-binding protein 2 (GATA2), chemokine (C—C motif) ligand 2 (CCL2), and RAS-interacting protein 1 (RASIP1) to promote angiogenesis.

12. The pharmaceutical composition for hair loss treatment comprising immune-tolerized fetal stem cell-derived extracellular vesicles according to claim 10, wherein the extracellular vesicles additionally contain miR-29, miR-203, and miR-218 to suppress DKK1 gene expression.

13. The pharmaceutical composition for hair loss treatment comprising immune-tolerized fetal stem cell-derived extracellular vesicles according to claim 10, wherein the extracellular vesicles are established by culturing fetal stem cells isolated from amniotic fluid collected for amniocentesis in early pregnancy in an ex vivo culture matrix that induces immune tolerance properties.

14. A cosmetic composition for hair regeneration and hair loss alleviation comprising immune-tolerized fetal stem cell-derived extracellular vesicles that are manufactured by the method according to claim 1, wherein
the extracellular vesicles contain noggin, sclerostin domain-containing protein 1 (SOSTDC1), fibroblast growth factor-7 (FGF-7), and platelet-derived growth factor-alpha (PDGF-α) to promote conversion of hair in the catagen phase into hair in the anagen phase.
